(19) United States
(12) Patent Application Publication
(10) Pub. No.: US 2023/0210911 A1
(43) Pub. Date: Jul. 6, 2023

(54) METHODS AND MATERIALS FOR TREATING NON-MALIGNANT DISORDERS OR DISEASES WITH CORD BLOOD

(71) Applicant: University of Pittsburgh- Of the commonwealth System of Higher Education, Pittsburgh, PA (US)

(72) Inventor: Paul Szabolcs, Sewickley, PA (US)

(21) Appl. No.: 18/008,482

(22) PCT Filed: Jun. 16, 2021
(86) PCT No.: PCT/US2021/037560

§ 371 Date: Dec. 6, 2022

Related U.S. Application Data
(60) Provisional application No. 63/044,868, filed on Jun. 26, 2020.

(51) Int. Cl. A61K 35/51; A61K 9/00; A61P 37/00

(52) U.S. Cl. CPC A61K 35/51; A61K 9/0019; A61P 37/00

(57) ABSTRACT

This document provides methods and materials for using umbilical cord blood to treat non-malignant disorders. For example, methods and materials for administering umbilical cord blood to a mammal (e.g., a human) via multiple (e.g., two or more) infusions to treat non-malignant diseases such as inherited disorders of metabolism, immunity, and/or hematopoiesis are provided.

### CROSS-REFERENCE TO RELATED APPLICATIONS

This application claims the benefit of U.S. Provisional Application Ser. No. 63/044,868, filed Jun. 26, 2020. The disclosure of the prior application is considered part of (and is incorporated by reference in) the disclosure of this application.

### STATEMENT AS TO FEDERALLY SPONSORED RESEARCH

This invention was made with government support under HL091749 awarded by National Institutes of Health. The government has certain rights in the invention.

### BACKGROUND

### 1. Technical Field

This document relates to methods and materials for using umbilical cord blood (e.g. umbilical cord blood obtained from a cord blood bank) to treat non-malignant disorders or diseases. For example, umbilical cord blood can be administered to a mammal (e.g., a human) in multiple (e.g., two or more) infusions to treat non-malignant disorders or diseases such as inherited disorders of metabolism, immunity, and/or hematopoiesis.

### 2. Background Information

Myeloablative conditioning (MAC) treatments in combination with hematopoietic stem cell transplantation (HSCT) can ameliorate a broad spectrum of non-malignant disorders (NMD), including primary immunodeficiency diseases (PID), hemoglobinopathies, bone marrow failure syndromes, and inborn errors of metabolism (IEM) by replacing defective red blood cells or leukocytes. MAC given before an HSCT can include high-dose chemotherapy and/or radiation to knock down the immune system of a patient to allow a stem cell graft to take hold. The MAC causes irreversible cytopenia of two primary components of blood: erythrocytes (e.g., red blood cells) and leukocytes (e.g., white blood cells). Reduced intensity conditioning (RIC) regimens have been developed using different chemotherapeutic agents and/or lower doses of radiation to reduce the toxicity to tissues like lungs, liver, heart and even bone marrow.

The hematopoietic stem cells (HSCs) are multipotent stem cells from a type-matched donor and are transplanted via cord blood transfusion to reverse the cytopenia caused by the MAC or RIC. The HSCT engrafts the donor stem cells into the patient to create a functional immune system.

### SUMMARY

This document provides methods and materials for using umbilical cord blood to treat non-malignant disorders or diseases. For example, this document provides methods and materials for administering umbilical cord blood to a mammal (e.g., a human) via multiple (e.g., two or more) infusions to treat non-malignant disorders or diseases such as inherited disorders of metabolism, immunity, and/or hematopoiesis.

As demonstrated herein, umbilical cord blood can be obtained from a single donor preparation, and a portion (e.g., 80-95 percent of the original total) of that preparation can be administered to the recipient during a first time point. In addition, some or all of the portion (e.g., 5-20 percent of the original total) not initially administered to the recipient can be stored (e.g., refrozen and stored) for use in a second or subsequent administration to that same recipient at a second time point that is, for example, two weeks to eight months (e.g., one to three months) after the first time point.

Before the initial administration of a portion of the umbilical cord blood preparation, the mammal (e.g., a human having a non-malignant disorder) can be administered a MAC or RIC regimen to knock down the recipient's immune system so that the recipient can adopt the donor's immune system via the cells (e.g., hematopoietic stem cells) present in the umbilical cord blood preparation. In some cases, this transplantation can be less effective than desired. For example, toxic agents (e.g., lymphotoxic agents such as alemtuzumab or anti-thymocyte globulin (ATG)) from the MAC or RIC regimen remaining within the recipient (or administered during the post-transplantation phase) may reduce or slow effective establishment of immune cell populations (e.g., lymphocyte populations) within the recipient, making that recipient potentially susceptible to adverse events such as infections (e.g., bacterial, fungal, and/or viral infections). To help the recipients of an initial administration of a portion of an umbilical cord blood preparation establish the donor's immune system quicker, more effectively, and/or with reduced adverse events, the recipient can be administered one or more subsequent administrations of a remaining, refrozen portion of that same umbilical cord blood preparation initially administered two weeks to eight months after that initial administration. In some cases, the one or more subsequent administrations can provide the transplant recipient with an immune boost at a time when the initially administered umbilical cord blood has yet to establish an effective immune system within the recipient. This vulnerable period is typically within the first three months after cord blood transplant. The immune boost is optimally thawed and infused at a time when the concentrations of toxic agent(s) (e.g., alemtuzumab and/or ATG) that are part of the MAC or RIC regimen decline within the recipient to minimal or zero, thereby allowing the pre-existing immune cells (e.g., leukocytes and lymphocytes) present in the umbilical cord blood preparation to survive and function within the recipient. In some cases, such a subsequent administration of a refrozen portion of the same umbilical cord blood preparation initially administered to a recipient can be referred to herein as a cord donor lymphocyte infusion (cDLI).

In general, one aspect of this document features a method for providing a mammal with a hematopoietic stem cell transplantation. The method comprises (or consists essentially of or consists of) (a) administering a first portion of an umbilical cord blood preparation obtained from a single donor to the mammal at a first time point, and (b) administering a second portion of the umbilical cord blood preparation to the mammal at a second time point, wherein the second time point is two weeks to eight months after the first time point. The mammal can be a human. The first portion can comprise from about 80 to about 95 percent of the umbilical cord blood preparation. The first portion can comprise from about 90 to about 95 percent of the umbilical cord blood preparation. The first portion can be a portion that was obtained from the umbilical cord blood preparation after the umbilical cord blood preparation was thawed from a frozen state only once. The method can comprise refreezing a remaining portion of the umbilical cord blood preparation that remains after the first portion is obtained for the administering step (a). The second portion can comprise from about 5 to about 20 percent of the umbilical cord blood preparation. The second portion can comprise from about 5 to about 10 percent of the umbilical cord blood preparation. The second portion can be a portion that was obtained from the umbilical cord blood preparation after the umbilical cord blood preparation was thawed from a frozen state twice. The administering step (a) can be via an intravenous injection. The administering step (b) can be via an intravenous injection. The method can comprise administering alemtuzumab, ATG, hydroxyurea, fludarabine, melphalan, thiotepa, or a combination thereof to the mammal prior to the administering step (a). The second time point can be one month to six months after the first time point. The second time point can be one month to three months after the first time point.

In another aspect, this document features a method for treating a mammal having a non-malignant disorder with a hematopoietic stem cell transplantation. The method comprises (or consists essentially of or consists of) (a) administering a first portion of an umbilical cord blood preparation obtained from a single donor to the mammal at a first time point, and (b) administering a second portion of the umbilical cord blood preparation to the mammal at a second time point, wherein the second time point is two weeks to eight months after the first time point. The mammal can be a human. The non-malignant disorder can be a primary immunodeficiency disease, a hemoglobinopathy, a bone marrow failure syndrome, or an inborn errors of metabolism disorder. The first portion can comprise from about 80 to about 95 percent of the umbilical cord blood preparation. The first portion can comprise from about 90 to about 95 percent of the umbilical cord blood preparation. The first portion can be a portion that was obtained from the umbilical cord blood preparation after the umbilical cord blood preparation was thawed from a frozen state only once. The method can comprise refreezing a remaining portion of the umbilical cord blood preparation that remains after the first portion is obtained for the administering step (a). The second portion can comprise from about 5 to about 20 percent of the umbilical cord blood preparation. The second portion can comprise from about 5 to about 10 percent of the umbilical cord blood preparation. The second portion can be a portion that was obtained from the umbilical cord blood preparation after the umbilical cord blood preparation was thawed from a frozen state twice. The administering step (a) can be via an intravenous injection. The administering step (b) can be via an intravenous injection. The method can comprise administering alemtuzumab, ATG, hydroxyurea, fludarabine, melphalan, thiotepa, or a combination thereof to the mammal prior to the administering step (a). The second time point can be one month to six months after the first time point. The second time point can be one month to three months after the first time point.

In another aspect, this document features a method for treating a mammal having a non-malignant disorder with a hematopoietic stem cell transplantation. The method comprises (or consists essentially of or consists of) (a) thawing a frozen umbilical cord blood preparation obtained from a single donor, thereby obtaining a thawed umbilical cord blood preparation, (b) administering a first portion of the thawed umbilical cord blood preparation to the mammal at a first time point, (c) re-freezing a remaining portion of the thawed umbilical cord blood preparation that remains after the first portion was used for the administering step (b), thereby obtaining a refrozen umbilical cord blood preparation, (d) thawing the refrozen umbilical cord blood preparation, thereby obtaining a twice thawed umbilical cord blood preparation, and (e) administering a portion of the twice thawed umbilical cord blood preparation to the mammal at a second time point, wherein the second time point is two weeks to eight months after the first time point. The mammal can be a human. The non-malignant disorder can be a primary immunodeficiency disease, a hemoglobinopathy, a bone marrow failure syndrome, or an inborn errors of metabolism disorder. The first portion can comprise from about 80 to about 95 percent of the frozen umbilical cord blood preparation. The first portion can comprise from about 90 to about 95 percent of the frozen umbilical cord blood preparation. The second portion can comprise from about 5 to about 20 percent of the frozen umbilical cord blood preparation. The second portion can comprise from about 5 to about 10 percent of the frozen umbilical cord blood preparation. The administering step (a) can be via an intravenous injection. The administering step (b) can be via an intravenous injection. The method can comprise administering alemtuzumab, ATG, hydroxyurea, fludarabine, melphalan, thiotepa, or a combination thereof to the mammal prior to the thawing step (a). The second time point can be one month to six months after the first time point. The second time point can be one month to three months after the first time point.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1. Quantification of graft neutrophil engagement in patients in days post-transplant. (A) Neutrophil engraftment was defined as the first of three consecutive days with an absolute neutrophil count of >500 cells/μL. Neutrophil engraftment occurred in all patients at a median of day 15 days post-transplant (range: 10-33 days). (B) Platelet engraftment was defined as the first of 7 consecutive days with a platelet count >20,000 cells/μL or >50,000 cells/μL without transfusion support. Platelet engraftment >20,000 cells/μL occurred in all evaluable patients at a median of 32 days post-transplant (range: 15-56 days) and platelet engraftment >50,000 cells/μL occurred in all evaluable patients at a median of 37 days post-transplant (range: 27-79 days).

FIG. 2. Donor chimerism was measured by STR analysis in peripheral whole blood and, when sufficient CD3⁺ cells were present in peripheral blood, in sorted CD3⁺ cells. Donor chimerism of >95% in whole blood was seen in 81%, 83%, 76%, and 65% of patients and in 71%, 53%, 63%, and 71% for CD3⁺ cells at day 100, 180 and 1 and 2 years post-transplant, respectively.

FIG. 3. Quantification of GVHD. (A) The cumulative incidence of grade II-IV GVHD was 27% (95% CI: 10-48%) and the cumulative incidence of grade III-IV GVHD was 11% (95% CI: 1-40%). The median onset day for grade II-IV GVHD was 40 days post-transplant (range: 14-145 days). (B) The cumulative incidence of limited chronic GVHD was 42% (95% CI: 25-58%). No patients developed severe or extensive chronic GVHD.

FIG. 4. Quantification of survival. (A) Overall survival was 95% (95% CI: 83-99%) at 1 year and EFS was 93% (95% CI: 80-98%) at 1 year. (B) Overall survival was 85% (95% CI: 67-94%) at 5 years post-transplant and 83% (95% CI: 66-92%) at 5 years post-transplant.

FIG. 5. Quantification of immune reconstitution post-transplant. (A-F) Absolute numbers of CD3, CD4, CD8, CD4/CD45RA/CD62L⁺, CD19, and CD16/56 cells as measured by flow cytometry are shown. Each box extends from the 25th to 75th percentile with a line at the median and whiskers extending 1.5 times the interquartile range.

FIG. 6. Quantification of the number of T-cell receptor excision circles (TREC). TREC normalized per 1×10⁶ CD3 cells and T-cell receptor (TCR) V3 repertoire, measured as TCR spectrotype complexity score (TCR SCS), which counts the number of peaks/TCR V3 subfamily, were measured pre- and post-transplant.

FIG. 7. An exemplary representation of pre- and post-transplant TCR SCS.

FIG. 8. Quantification of alemtuzumab levels. Day 0 alemtuzumab levels were measured in 26 subjects. The median alemtuzumab concentration was 0.58 μ/mL (range: 0-2.82 μg/mL) at day 0.

FIG. 9. Quantification of immune response. (A-C) Subjects with alemtuzumab above the median had lower absolute CD3, CD4, and CD8 counts at 30 and 60 days post-transplant when compared to subjects with alemtuzumab levels below the median (*=p<0.05; **=p <0.01).

FIG. 10. Quantification of cDLI effect post-transplant. (A) Nine subjects received cDLI for mixed whole blood or T cell chimerism. Improvement in T cell chimerism was seen in the majority of patients at 120 days after cDLI. (B) Ten subjects received cDLI for viral infection (seven with viremia; three with symptomatic viral gastroenteritis). Improvement in viremia and/or clinical symptoms was seen in 6 of 10 subjects.

FIG. 11. Quantification of viral copy number after cDLI through 60 days post-transplant.

FIG. 12. Quantification of immune reconstitution in CD3 (cells/μL) after cDLI through 60 days post-transplant.

FIG. 13. Quantification of cumulative incidence of acute GVHD or progression of acute GVHD in patients who did or did not receive cDLI.

FIG. 14. Conditioning regimen and alemtuzumab dosing strata.

FIG. 15. Quantification of neurocognitive outcomes for symptomatic infantile-onset Krabbe disease. Cognitive outcomes are shown by plotting age-equivalent score (developmental age) against actual age (calendar age) for symptomatic infantile Krabbe patients. Red and blue lines indicate individual values of children who received RIC (red) and historical MAC (blue). Gray lines represent the mean and approximate variability (95% CI) observed in the normal population.

FIG. 16. Quantification of immune reconstitution post-transplant. Immune reconstitution measured as the percent of age-appropriate normal values of each lymphocyte subset achieved at each time point. Normal values of lymphocyte subsets were defined as an absolute cell count of ≥10th percentile of those seen in healthy children of the same age at the time of measurement.

FIG. 17. Quantification of infection post-transplant. The cumulative incidence of first infection after initiation of conditioning was 61% (95% CI: 49-72%), and the first infection was detected at a median of 18 days post-transplant (range: −27-224) post-transplant.

FIG. 18. Quantification of correlation of alemtuzumab level with GVHD, infection, and chimerism. (A) The cumulative incidence of grade II-IV GVHD in patients with low day 0 alemtuzumab was 23% compared to 15% in those with high day 0 alemtuzumab levels (p=0.5). (B) The cumulative incidence of first viral infection in patients with both low and high day 0 alemtuzumab levels was 54% (p-0.9). (C) The median donor percentage in patients with low and high day 0 alemtuzumab levels was 100% donor in both whole blood and T cell fractions (p=0.35 and p=0.94, respectively).

FIG. 19. Chart depicting day of cDLI post-transplant. (A) The distribution of the day of cDLI infusion post-transplant is shown. The median day of infusion was day 66 post-transplant. Error bars indicate the interquartile range. Quantification of kinetics of immune reconstitution. (B-D) The kinetics of T cell reconstitution for subjects who received cDLI between day 38 and 75 post-transplant (N=14) are shown in red, while those who never received cDLI (N=24) are shown in blue. The inset shows the absolute T cell numbers at day 60 and 100 post-transplant. All points represent median absolute values with error bars indicating the interquartile range. The lines between points represent the best-fit line using simple linear regression.

### DETAILED DESCRIPTION

This document provides methods and materials for using umbilical cord blood to treat non-malignant disorders or diseases. For example, this document provides methods and materials for administering umbilical cord blood to a mammal (e.g., a human) via multiple (e.g., two or more), appropriately timed administrations (e.g., infusions) to treat non-malignant disorders or diseases.

Any appropriate mammal can be treated as described herein. For example, humans and other primates such as monkeys having one or more non-malignant disorders or diseases can be treated using umbilical cord blood as described herein. In some cases, dogs, cats, horses, cows, pigs, sheep, rabbits, mice, and rats having one or more non-malignant disorders or diseases can be treated using umbilical cord blood as described herein.

When treating a human having a non-malignant disorder or disease as described herein, that human can be any appropriate age (e.g., from 0.1 years to 60 years of age). For example, a human having a non-malignant disorder or disease and treated using umbilical cord blood as described herein can be a human that is less than 30 years of age. In some cases, the human having a non-malignant disorder or disease and treated using umbilical cord blood as described herein can be a human that is from one month old to 30 years old (e.g., from one month old to 20 years old, from one month old to 15 years old, from three months old to 30 years old, from three months old to 20 years old, from three months old to 15 years old, from six months old to 30 years old, from six months old to 20 years old, from six months old to 15 years old, from six months old to 12 years old, from six months old to ten years old, from six months old to five years old, from six months old to three years old, from one year old to 15 years old, from one year old to 12 years old, from one year old to ten years old, from one year old to five years old, from one year old to three years old, or from one year old to two years old).

Any appropriate non-malignant disorder or disease can be treated as described herein. For example, hematologic diseases, metabolic disorders, environmentally-induced diseases, viral diseases, autoimmune diseases, lysosomal storage disorders (e.g., congenital lysosomal storage disorders), immunodeficiencies (e.g., congenital immunodeficiencies), and hematologic diseases (e.g., congenital hematologic diseases) can be treated via multiple (e.g., two or more), appropriately timed administrations (e.g., infusions) of umbilical cord blood as described herein. Examples of hematologic diseases that can be treated as described herein include, without limitation, phagocyte disorders (e.g., myelodysplasia), anemias (e.g., paroxysmal nocturnal hemoglobinuria (PNH; severe aplasia), aplastic anemia, and acquired pure red cell aplasia), and myeloproliferative disorders (e.g., polycythemia vera, essential thrombocytosis, and myelofibrosis). Examples of metabolic disorders that can be treated as described herein include, without limitation, amyloidoses (e.g., amyloid light chain (AL) amyloidosis). An example of an environmentally-induced disease that can be treated as described herein includes, without limitation, radiation poisoning. Examples of viral diseases that can be treated as described herein include, without limitation, human T-lymphotropic virus (HTLV) infections and human immunodeficiency virus (HIV) infections. An example of an autoimmune disease that can be treated as described herein includes, without limitation, multiple sclerosis. Examples of lysosomal storage disorders that can be treated as described herein include, without limitation, (1) lipidoses disorders such as (a) neuronal ceroid lipofuscinoses (e.g., infantile neuronal ceroid lipofuscinosis (INCL, Santavuori disease) and Jansky-Bielschowsky disease (late infantile neuronal ceroid lipofuscinosis)), (b) sphingolipidoses (e.g., Niemann-Pick disease and Gaucher disease), and (c) leukodystrophies (e.g., adrenoleukodystrophy, metachromatic leukodystrophy, and Krabbe disease (globoid cell leukodystrophy)), (2) mucopolysaccharidoses such as Hurler syndrome (MPS I H, α-L-iduronidase deficiency), Scheie syndrome (MPS I S), Hurler-Scheie syndrome (MPS I H-S), Hunter syndrome (MPS II, iduronidase sulfate deficiency), Sanfilippo syndrome (MPS III), Morquio syndrome (MPS IV), Maroteaux-Lamy syndrome (MPS VI), and Sly syndrome (MPS VII), (3) glycoproteinoses such as mucolipidosis II (I-cell disease), fucosidosis, aspartylglucosaminuria, and alpha-mannosidosis, and (4) Wolman disease (acid lipase deficiency). Examples of immunodeficiencies that can be treated as described herein include, without limitation, (1) T-cell deficiencies such as ataxia-telangiectasia and DiGeorge syndrome, (2) combined T- and B-cell deficiencies such as severe combined immunodeficiency (SCID) of any type, (3) Wiskott-Aldrich syndrome, (4) phagocyte disorders such as Kostmann syndrome and Shwachman-Diamond syndrome, (5) immune dysregulation diseases such as Griscelli syndrome, type II, and (6) innate immune deficiencies such as NF-Kappa-B essential modulator (NEMO) deficiency (e.g., inhibitor of Kappa Light polypeptide gene enhancer in B Cells gamma kinase deficiency). Examples of hematologic diseases that can be treated as described herein include, without limitation, (1) hemoglobinopathies such as sickle cell disease and p thalassemia major (e.g., Cooley's anemia), (2) anemias such as aplastic anemia (e.g., Diamond-Blackfan anemia and Fanconi anemia), (3) cytopenias such as amegakaryocytic thrombocytopenia, and (4) hemophagocytic syndromes such as hemophagocytic lymphohistiocytosis (HLH). In some cases, a human having a primary immunodeficiency disease (PID), a hemoglobinopathy, a bone marrow failure syndrome, autoimmune disorders, or an inborn error of metabolism (IEM) can be treated as described herein.

Before the first administration of umbilical cord blood as described herein, the recipient mammal (e.g., human patient) can be exposed to myeloablative conditioning (MAC) or reduced intensity conditioning (RIC) to knock down the recipient's immune system in a manner that promotes the ability of the hematopoietic stem cells of the to-be-administered umbilical cord blood to reconstitute the donor's immune system or a chimeric donor/recipient immune system within the recipient. Any appropriate MAC or RIC can be used to prepare the mammal to receive the donor umbilical cord blood. For example, an MAC or RIC regimen can include administering alemtuzumab, ATG, hydroxyurea, fludarabine, melphalan, thiotepa, or a combination thereof to the mammal. In some cases, an MAC regimen as set forth in Table 1 can be administered to the mammal. In some cases, an MAC regimen can be administered to the mammal as described elsewhere (Escolar et al., N. Engl. J. Med., 352(20): 2069-81 (2005); Boelens et al., Biology of blood and marrow transplantation: J. Am. Soc. Blood Marrow Trans., 15(5):618-25 (2009); Baronciani et al., Bone Marrow Trans., 51(4):536-41 (2016); van den Broek et al., Blood Adv., 2(1):49-60 (2018); and Prasad et al., Blood, 112(7):2979-89 (2008)). In some cases, an RIC regimen as set forth in Table 2 can be administered to the mammal. In some cases, an RIC regimen can be administered to the mammal as described elsewhere (Satwani et al., Bone Marrow Trans., 41(2):173-82 (2008); Amrolia et al., Blood, 96(4):1239-46 (2000); Rao et al., Blood, 105(2):879-85 (2005); Parikh et al., Biology of blood and marrow transplantation: J. Am. Soc. Blood Marrow Trans., 20(3):326-36 (2014); Allen et al., Blood, 132(13):1438-51 (2018); Lindemans et al., Blood, 123(1):126-32 (2014); and Gungor et al., Lancet, 383(9915):436-48 (2014)).

TABLE 1
Exemplary myeloablative conditioning (MAC) protocols.

| Drug/Treatment | Duration | Dose |
|---|---|---|
| FLU/BU REGIMENS: | Bu administered over the | Cumulative Bu: |
| Fludarabine (FLU) + Busulfan (BU) + | course of 4 days | 12-16 mg/kg |
| either Alemtuzumab (aka CAMPATH) or | | |
| anti-thymocyte globulin (ATG) | | |
| BU/CY REGIMENS: | Bu administered over the | Cumulative BU: |
| Busulfan + Cytoxan (Cy) + | course of 3-4 days; Cy given | 14-16 mg/kg range; |
| either Alemtuzumab or ATG | over 2-4 days | Cumulative Cy: |
| | | 120-200 mg/kg range |
| Reduced Toxicity MAC: | Bu administered over the | Cumulative Bu: |
| Fludarabine + Busulfan (BU) + | course of 2-3 days | 9-12 mg/kg range |
| either Alemtuzumab or ATG | | |

TABLE 2
Exemplary reduced intensity conditioning (RIC) protocols.

| Drug/Treatment | Duration | Dose |
|---|---|---|
| Fludarabine + Busulfan + | Fludarabine over the course | Fludarabine |
| either Alemtuzumab or ATG | of 4-5 days; Busulfan over | 25-30 mg/msq/dose |
| | the course of 3-4 days | or 1 mg/kg/dose; |
| | | Cumulative Bu: |
| | | ~8 mg/kg over 3-4 days |

In addition, any appropriate umbilical cord blood preparation can be selected for the particular mammal (e.g., the particular human) being treated. For example, a transplant clinician can assess the recipient mammal to identify an appropriate allogenic donor based on, for example, acceptable matching of human leukocyte antigen (HLA) types. Such matching can be performed as described in Example 1. In some case, HLA matching and selection of an appropriate umbilical cord blood preparation for a particular mammal (e.g., a particular human) can be performed as described elsewhere (Dehn et al., Blood, 134(12):924-934 (2019)).

An umbilical cord blood preparation can be obtained from any appropriate provider. For example, an umbilical cord blood preparation can be obtained from a private or public cord blood bank such as Carolinas Cord Blood Bank, BloodworksNW, LifebankUSA, StemCyte, CellSure, the Anthony Nolan Cord Blood Bank, NHS Cord Blood Bank, DKMS Cord Blood Bank, German Cord Blood Bank, Bavarian Stem Cell Bank, or New York Cord Blood.

In general, umbilical cord blood is collected from the fetal end of the cord within about ten minutes of birth. Typically, about 75±25 mL of cord blood is obtained per umbilical cord. In some cases, additional stem cells can be collected from the placenta. In some cases, testing for viruses (e.g., HIV and/or Hepatitis B or C), bacteria, and/or fungal organisms, HLA typing, cell count and/or cell viability analyses, and/or ABO and Rh blood grouping can be performed before the umbilical cord blood is frozen (e.g., cryopreserved) and stored.

Once the fresh umbilical cord blood is obtain, it can be processed by optionally removing red blood cells and then frozen (e.g., cryopreserved). In general, a cryopreservant such as dimethyl sulfoxide (DMSO) is added to the cord blood to help the cells survive the cryogenic process, which typically involves slowly cooling the cord blood to −90° C. At that point, the frozen umbilical cord blood preparation can be stored in liquid nitrogen (e.g., at −196° C.) until ready for use.

After the recipient mammal (e.g., a human) is prepared to receive the umbilical cord blood, the appropriate once-frozen umbilical cord blood preparation can be stored in the transplant center until day 0 and thawed within minutes. Briefly, cord blood banks typically ship cord blood units in the frozen state, and the typical volume is 25-50 mL frozen cord blood representing 100 percent of what the transplant center receives. This can be stored frozen until day 0 and then thawed and diluted into 100-110 mL total volume by mixing into a diluent solution. This diluent solution can be prepared in advance and placed in the refrigerator first thing in the morning on the day of transplant. The diluent solution can be prepared by mixing 250 mL of 10% LMD (low molecular dextran 40)+50 mL of 25% albumin.

Then, as described herein, a first portion can be obtained from the thawed umbilical cord blood preparation. Any appropriate portion can be obtained from the thawed umbilical cord blood preparation for an initial administration to the recipient mammal (e.g., a human) as a first portion. For example, from about 50 percent to about 97 percent (e.g., from about 50 percent to about 95 percent, from about 60 percent to about 95 percent, from about 70 percent to about 95 percent, from about 75 percent to about 95 percent, from about 80 percent to about 95 percent, from about 85 percent to about 95 percent, from about 90 percent to about 95 percent, from about 90 percent to about 98 percent, from about 90 percent to about 97 percent, from about 95 percent to about 98 percent, or from about 95 percent to about 97 percent) of the thawed umbilical cord blood preparation can be obtained as a first portion for the initial administration to the recipient mammal (e.g., a human).

As described herein, the remaining portion of the thawed umbilical cord blood preparation after the first portion is removed can be refrozen to form a twice-frozen umbilical cord blood preparation for a subsequent administration to that same recipient. For example, when 50 percent of the once thawed umbilical cord blood preparation is obtained as a first portion, the remaining 50 percent can be refrozen to form a twice-frozen umbilical cord blood preparation for future administration to that same recipient. In another example, when 95 percent of the once thawed umbilical cord blood preparation is obtained as a first portion, the remaining 5 percent can be refrozen to form a twice-frozen umbilical cord blood preparation for future administration to that same recipient. In some cases, the remaining portion can be refrozen and stored for future administration to that same recipient as a second portion that is from about 2 percent to about 50 percent (e.g., from about 5 percent to about 50 percent, from about 5 percent to about 40 percent, from about 5 percent to about 30 percent, from about 5 percent to about 25 percent, from about 5 percent to about 20 percent, from about 5 percent to about 15 percent, from about 5 percent to about 10 percent, from about 3 percent to about 15 percent, from about 3 percent to about 10 percent, from about 2 percent to about 10 percent, from about 2 percent to about 5 percent, from about 2 percent to about 4 percent, or from about 3 percent to about 4 percent) of the total original once-frozen umbilical cord blood preparation.

In some cases, the refrozen aliquot is prepared by taking 5 mL of 100 to 110 mL of thawed and diluted cord blood. This aliquot can be combined with 7.0 mL of freeze mix (e.g., 5.8 mL 0.9% NaClI/1.2 mL DMSO) for a total volume of 10 mL (+2 mL for sterility testing) and frozen per standard procedures for Cryopreservation of Human Hematopoietic Progenitor Cells. Then, this product can be reserved (e.g., maintained frozen) for future cDLI as described herein, if needed.

In some case, the first portion of thawed umbilical cord blood preparation can be processed before being administered to the recipient mammal (e.g., a human). For example, the entire contents of the original once-frozen umbilical cord blood preparation as obtained from the cord blood bank can be thawed and washed. The washing step can be performed by resuspending the unit into 100 mL Plasma-lyte A and followed centrifugation. At this point, the pelleted cells from the washing step can be resuspended in any appropriate solution (e.g., cold dextran 40/albumin mixtures such as the diluent solution described above, Plasma Lyte A, or normal saline supplemented with human albumin with a final concentration of 1-5 percent) to any appropriate volume. For example, the pelleted cells can be resuspended to a volume that is from 10 mL to 200 mL (e.g., from 10 mL to 150 mL, from 10 mL to 100 mL, from 25 mL to 200 mL, from 50 mL to 200 mL, from 50 mL to 150 mL, from 75 mL to 125 mL, or from 80 mL to 120 mL). In some cases, the pelleted cells can be resuspended to a volume of 100 mL. In this example, since the entire contents were processed, the first portion can be obtained from the resuspended cells. For example, when the pelleted cells are resuspended to a volume of 100 mL and the first portion is designed to be 95 percent of the total original once-frozen umbilical cord blood preparation, 95 mL of that volume can be obtained as a first portion for administration to the recipient mammal (e.g., a human). In this case, the remaining 5 mL can be re-frozen for subsequent use as a second portion.

In some cases, the first portion of thawed umbilical cord blood preparation can be administered to the recipient mammal (e.g., a human) without performing a post-thaw washing step. For example, the entire contents of the original once-frozen umbilical cord blood preparation as obtained from the cord blood bank can be thawed and diluted to any appropriate volume as noted above (e.g., to 100 mL). In this case, when the volume of the thawed umbilical cord blood preparation is diluted to 100 mL and the first portion is designed to be 95 percent of the total original once-frozen umbilical cord blood preparation, 95 mL of that volume can be obtained as a first portion for administration to the recipient mammal (e.g., a human). In this case, the remaining 5 mL can be re-frozen for subsequent use as a second portion.

In some case, the first portion can be removed from the thawed umbilical cord blood preparation, and the remaining portion can be re-frozen for subsequent use as a second portion. In this case, the removed first portion can be processed (e.g., with or without post-thaw washing) as described above before being administered to the recipient mammal (e.g., a human). In some case, the second portion can be removed from the thawed umbilical cord blood preparation and re-frozen for subsequent use as a second portion, and the remaining portion can be used as a first portion for initial administration to the recipient mammal (e.g., a human). In this case, the remaining first portion can be processed (e.g., with or without post-thaw washing) as described above before being administered to the recipient mammal (e.g., a human).

Any appropriate method can be used to administer the first portion of the umbilical cord blood preparation to the recipient mammal (e.g., a human) as the initial administration of cord blood to that recipient mammal. For example, the first portion can be administered to the recipient mammal (e.g., a human) via an intravenous infusion or an intrabone injection (e.g., an intrabone injection into the hip in 4 to 6 independent aliquots of about 3 to 5 mL each). In some cases, the first portion of the umbilical cord blood preparation can be infused over a time period from 15 minutes to 90 minutes (e.g., from 15 minutes to 75 minutes, from 15 minutes to 60 minutes, from 30 minutes to 90 minutes, from 45 minutes to 90 minutes, from 30 minutes to 75 minutes, or from 45 minutes to 60 minutes).

The administration of the first portion of the umbilical cord blood preparation to the recipient mammal (e.g., a human) as the initial administration of cord blood to that recipient mammal can be designated as day 0 or the day of initial cord blood transfusion. As described herein, a second portion of the same umbilical cord blood preparation can be administered to the same recipient mammal (e.g., a human) at a second time point that is subsequent to the time point of the initial administration (i.e., subsequent to day 0 or subsequent to the day of initial cord blood transfusion). This second time point can be any appropriate time point from the first time point (i.e., day 0). For example, the second time point can be from two weeks to one year (e.g., from three weeks to one year, from one month to one year, from six weeks to one year, from two months to one year, from three months to one year, from four months to one year, from five months to one year, from six months to one year, from seven months to one year, from eight months to one year, from nine months to one year, from two weeks to eleven months, from two weeks to ten months, from two weeks to nine months, from two weeks to eight months, from two weeks to seven months, from two weeks to six months, from two weeks to five months, from two weeks to four months, from two weeks to three months, from two weeks to two months, from two weeks to one month, from one month to ten months, from one month to nine months, from one month to eight months, from six weeks to ten months, from six weeks to nine months, from six weeks to eight months, from two months to ten months, from two months to nine months, from two months to eight months, three months to eight months, or one month to three months) after the first time point (i.e., day 0).

The twice-frozen umbilical cord blood preparation can be thawed and processed to obtain a second portion for administration in a manner similar to that described herein for the first portion. For example, the twice-frozen umbilical cord blood preparation can be thawed, optionally washed, and diluted to any appropriate volume. In some cases, a twice-frozen umbilical cord blood preparation can be thawed and diluted (with or without washing) to a volume from 10 mL to 200 mL (e.g., from 10 mL to 150 mL, from 10 mL to 100 mL, from 25 mL to 200 mL, from 50 mL to 200 mL, from 50 mL to 150 mL, from 75 mL to 125 mL, or from 80 mL to 120 mL). In some cases, since the total volume of the twice-frozen umbilical cord blood preparation may be less than the total volume of the original once-frozen umbilical cord blood preparation given the use of the first portion for the initial administration to the recipient mammal (e.g., a human), the twice-frozen umbilical cord blood preparation can be thawed and diluted to a volume from 5 mL to 50 mL (e.g., from 5 mL to 45 mL, from 5 mL to 40 mL, from 5 mL to 35 mL, from 5 mL to 30 mL, from 10 mL to 50 mL, from 15 mL to 50 mL, from 20 mL to 50 mL, from 25 mL to 50 mL, or from 25 mL to 35 mL). For example, a 10 to 12 mL twice-frozen umbilical cord blood preparation can be thawed and diluted with a diluent solution described above to a final volume of about 30 to 35 mL.

Any appropriate method can be used to administer the second portion of the umbilical cord blood preparation to the recipient mammal (e.g., a human) that received an initial administration of that same umbilical cord blood preparation as a first portion. For example, the second portion can be administered to the recipient mammal (e.g., a human) via an intravenous infusion or an intrabone injection (e.g., an intrabone injection into the hip in 4 to 6 independent aliquots of about 3 to 5 mL each). In some cases, the second portion of the umbilical cord blood preparation can be infused over a time period from 15 minutes to 90 minutes (e.g., from 15 minutes to 75 minutes, from 15 minutes to 60 minutes, from 30 minutes to 90 minutes, from 45 minutes to 90 minutes, from 30 minutes to 75 minutes, or from 45 minutes to 60 minutes).

As described herein, the second portion can be administered to the recipient mammal (e.g., a human) at a time point when the recipient would benefit from an immune boost. Such a time point can be when the amount of toxic agent(s) (e.g., alemtuzumab and/or ATG) from an MAC or RIC regime used to prepare the recipient mammal (e.g., a human) is less than (e.g., 10, 25, 50, 75, or 100 percent less than) the amount that was present in that recipient mammal at the time of the initial administration of the first portion (i.e., at day 0). In some cases, the time point for administering the second portion can be 5 to 30 days after the recipient mammal (e.g., a human) receives its last dose of a lymphotoxic agent such as alemtuzumab and/or ATG.

In some cases, a recipient mammal (e.g., a human) can undergo a de-escalation regime of one or more agents (e.g., one or more agents used in the MAC or RIC regime such as alemtuzumab and/or ATG) post-initial transfusion (e.g., post-day 0). In such cases, the second portion of the umbilical cord blood preparation can be administered to that recipient mammal during the de-escalation regime. In such cases, the second portion of the umbilical cord blood preparation can be administered to that recipient mammal at any time point after day +14.

In some cases, alemtuzumab can be administered with distal timing (e.g., at day −21, day −20, or day −19), with intermediate timing (e.g., at day −14 or day −13), or with proximal timing (e.g., at day −7, −6, −5, or −4) (see, e.g., Marsh et al., Biol. Blood Marrow Transplant, 21(8):1460-70 (2015)). Typically, the closer alemtuzumab is administered to day 0, the higher the alemtuzumab level will be on day 0. Thus, moving the timing further away from day 0 can result in lower alemtuzumab levels at day 0 and later, thereby allowing for sooner administration of cDLI. In general, the lowest dose of alemtuzumab used in any timing schedule can be 0.2 mg/kg/dose×1) with 3 mg/kg/total being the highest dose.

In some cases, the same recipient mammal (e.g., a human) can be administered more than two portions of the same umbilical cord blood preparation (e.g., more than a first portion and second portion of the same umbilical cord blood preparation). For example, in some cases, a recipient mammal (e.g., a human) can be administered three, four, or five portions of the same umbilical cord blood preparation at different time points. In one such example, a recipient mammal (e.g., a human) can be administered a first portion that is 90 percent of the original umbilical cord blood preparation at a first time point, a second portion that is 5 percent of the original umbilical cord blood preparation at a second time point subsequent to the first time point, and a third portion that is 5 percent of the original umbilical cord blood preparation at a third time point that is subsequent to the second time point.

The times between each of the three or more time points when more than two portions of the same umbilical cord blood preparation are administered to the same recipient can be any appropriate time points. For example, the second time point can be from two weeks to one year (e.g., from three weeks to one year, from one month to one year, from six weeks to one year, from two months to one year, from three months to one year, from four months to one year, from five months to one year, from six months to one year, from seven months to one year, from eight months to one year, from nine months to one year, from two weeks to eleven months, from two weeks to ten months, from two weeks to nine months, from two weeks to eight months, from two weeks to seven months, from two weeks to six months, from two weeks to five months, from two weeks to four months, from two weeks to three months, from two weeks to two months, from two weeks to one month, from one month to ten months, from one month to nine months, from one month to eight months, from six weeks to ten months, from six weeks to nine months, from six weeks to eight months, from two months to ten months, from two months to nine months, from two months to eight months, or three months to eight months) after the first time point (i.e., day 0), and the third time point can be from two weeks to one year (e.g., from three weeks to one year, from one month to one year, from six weeks to one year, from two months to one year, from three months to one year, from four months to one year, from five months to one year, from six months to one year, from seven months to one year, from eight months to one year, from nine months to one year, from two weeks to eleven months, from two weeks to ten months, from two weeks to nine months, from two weeks to eight months, from two weeks to seven months, from two weeks to six months, from two weeks to five months, from two weeks to four months, from two weeks to three months, from two weeks to two months, from two weeks to one month, from one month to ten months, from one month to nine months, from one month to eight months, from six weeks to ten months, from six weeks to nine months, from six weeks to eight months, from two months to ten months, from two months to nine months, from two months to eight months, or three months to eight months) after the second time point. When more the three portions of the same umbilical cord blood preparation are administered at different time points to the same recipient mammal, the subsequent portion(s) after the first three can be from two weeks to one year (e.g., from three weeks to one year, from one month to one year, from six weeks to one year, from two months to one year, from three months to one year, from four months to one year, from five months to one year, from six months to one year, from seven months to one year, from eight months to one year, from nine months to one year, from two weeks to eleven months, from two weeks to ten months, from two weeks to nine months, from two weeks to eight months, from two weeks to seven months, from two weeks to six months, from two weeks to five months, from two weeks to four months, from two weeks to three months, from two weeks to two months, from two weeks to one month, from one month to ten months, from one month to nine months, from one month to eight months, from six weeks to ten months, from six weeks to nine months, from six weeks to eight months, from two months to ten months, from two months to nine months, from two months to eight months, or three months to eight months) after the previous time point. In some cases, a first portion can be administered as day 0, a second portion can be administered one to three months after day 0, and a third portion can be administered one to three months after the second portion is administered.

In some cases, the recipient mammal (e.g., a human) can be monitored after the initial administration of a first portion, a second portion, or a subsequent portion (e.g., a third portion). For example, a recipient mammal (e.g., a human) can be monitored after the initial administration of a first portion for the presence or absence of CD3⁺ T lymphocytes (e.g., CD3⁺/CD4⁺ T cells and/or CD3⁺/CD8⁺ T cells). In some cases, circulating plasma levels of ATG preparations or alemtuzumab can be monitored to determine if the level has declined to a permissive range that is below the lympholytic cytotoxic range of about 0.1 μg/mL (see, e.g., Marsh et al., Blood, 127(4):503-512 (2016)). In some cases, a recipient mammal (e.g., a human) can be monitored after the administration of a second portion for the presence or absence of CD3⁺ T lymphocytes (e.g., CD3⁺/CD4⁺ T cells and/or CD3⁺/CD8⁺ T cells), CD16/56⁺ NK lymphocytes, and/or CD19⁺ B lymphocytes. As the absolute value of CD3⁺/CD4⁺ T cell counts rise over 100 cells/μL or over 200 cells/μL or over 400 cells/μL, which is normal for humans two years old or older, the probability for severe infections decreases. For example, when CD3⁺/CD4⁺ T cell counts are greater than 200 cells/μL, the probability for severe infections becomes negligible (e.g., less than 5 percent). If the CD3⁺/CD4⁺ T cell counts are unable to raise above 100 cells/μL, then additional measures may be needed such as tapering off immunosuppression medications. Recipient humans with values of less than 50 CD3⁺/CD4⁺ T cells/μL are particularly high risk for developing severe viral, opportunistic infections. The invention will be further described in the following examples, which do not limit the scope of the invention described in the claims.

### EXAMPLES

### Example 1—Reduced-Intensity Single-Unit Unrelated Cord Blood Transplant with Optional Immune Boost for Non-Malignant Disorders

HSCT from a healthy donor ameliorates non-malignant disorders including primary immunodeficiency diseases (PID), hemoglobinopathies, bone marrow failure syndromes, and inborn errors of metabolism (IEM) by replacing defective red blood cells or leukocytes or by releasing previously missing enzymes. Although MAC was initially employed to demonstrate efficacy, HSCT was demonstrated following RIC even in settings of partial host stem cell recovery, called mixed donor-recipient chimerism. RIC regimens also demonstrate decreased morbidity and treatment-related mortality (TRM) compared to MAC regimens; however, their widespread use has been limited by higher incidence of graft failure in chemotherapy-naïve patients undergoing unrelated cord blood transplant (UCBT) compared to those who have received prior chemotherapy. UCBT is suited for children with non-malignant disorders given its availability and absence of strict HLA matching requirements thus making UCBT possible for >95% of pediatric patients regardless of their ethnic background. Historically, a high proportion of patients who may benefit from allogeneic HSCT are not referred for transplantation as TRM for UCBT has remained in the range of 10-30% at one year for MAC and RIC regimens and many patients require a second transplantation. Procedure related morbidity may also limit referral from geneticists, hematologists, and immunologists, particularly for patients who exhibit advanced disease-specific symptoms.

### Patients

The first 44 consecutive patients with 20 genetically distinct non-malignant disorders (Table 3) underwent UCBT in a prospective clinical trial. Patients with severe combined immunodeficiency syndrome or chromosomal breakage syndromes were excluded as they would benefit from additional reductions in chemotherapy intensity. The first 15 patients received an identical chemotherapy regimen prior to registration. Because there was no difference in the conditioning regimen or patient characteristics (Table 4), all 44 patients were analyzed together. Informed consent was obtained from legal guardians of each patient. All patients had at least one year of follow-up, and all were chemotherapy-naïve.

TABLE 3
Comparison between Retrospective and Prospective Patients.

| | | Retrospective | | Prospective | | |
|---|---|---|---|---|---|---|
| | | N = 15 | | N = 29 | | p-value |
| Patient Characteristics | | | | | | |
| Age (yrs). - median (range) | | 1 | (0.5-14) | 3 | (0.4-16.6) | 0.19 |
| Weight (kg) - median (range) | | 10 | (7-52) | 15 | (6-74) | 0.33 |
| Gender - no (%) | Male | 8 | (53%) | 19 | (66%) | 0.52 |
| Diagnosis - no (%) | IMD | 8 | (53%) | 22 | (76%) | 0.07 |
| | PID | 6 | (40%) | 3 | (10%) | |
| | Other | 1 | (7%) | 4 | (14%) | |
| Graft Characteristics | | | | | | |
| HLA Match (of 6) | 6 | 7 | (47%) | 9 | (31%) | 0.28 |
| | 5 | 4 | (27%) | 15 | (52%) | |
| | 4 | 4 | (27%) | 5 | (17%) | |
| HLA Match (of 8) | 8 | 4 | (27%) | 4 | (14%) | 0.52 |
| | 7 | 2 | (13%) | 8 | (28%) | |
| | 6 | 4 | (27%) | 10 | (34%) | |
| | ≤5 | 5 | (33%) | 7 | (24%) | |
| TNC/kg (×10\hat{ }7) - median (range) | | 9.6 | (3.8-20.8) | 8.4 | (2.3-24.8) | 0.92 |
| CD34/kg (×10\hat{ }5) - median (range) | | 2.59 | (1.55-6.17) | 3.59 | (0.92-9.24) | 0.29 |
| Gender mismatch - no (%) | | 7 | (47%) | 15 | (52%) | >0.99 |
| ABO mismatch - no (%) | | 11 | (73%) | 18 | (62%) | 0.52 |
| Transplant Outcomes | | | | | | |
| Day Neutrophil engraftment - | | 15 | (12-33) | 15 | (10-21) | 0.80 |
| median (range) | | | | | | |
| 5-year overall survival - | | 87% | (56-97%) | 68% | (46-90%) | 0.75 |
| % (95% CI) | | | | | | |
| 5-year event-free survival - | | 87% | (56-97%) | 66% | (25-88%) | 0.56 |
| % (95% CI) | | | | | | |
| Transplant complications | | | | | | |
| Acute GVHD (Grade I-IV) - | | 40% | (13-67%) | 52% | (35-71%) | 0.49 |
| cumulative incidence (95% CI) | | | | | | |
| GVHD (Grade I-IV) onset day - | | 40 | (13-145) | 69 | (13-139) | 0.89 |
| median (range) | | | | | | |
| Acute GVHD (Grade II-IV) - | | 33% | (7-64%) | 24% | (12-44%) | 0.58 |
| cumulative incidence (95% CI) | | | | | | |
| GVHD (Grade II-IV) onset day - | | 47 | (21-145) | 28 | (14-110) | 0.43 |
| median (range) | | | | | | |
| Opportunistic Infections at | | 50% | (22-72%) | 60% | (42-79%) | 0.67 |
| 1 year - cumulative incidence | | | | | | |
| (95% CI) | | | | | | |

TABLE 4
Patient and Graft Characteristics.

| | | N = 44 | |
|---|---|---|---|
| Patient Characteristics | | | |
| Age (years) - median (range) | | 1.7 | (0.4-16.6) |
| Weight (kg) - median (range) | | 12 | (6-74) |
| Gender - no. (%) | Male | 27 | (61%) |
| Race - no (%) | White | 29 | (66%) |
| | African American | 8 | (18%) |
| | Hispanic | 3 | (7%) |
| | Asian | 1 | (2%) |
| | Alaska Native | 1 | (2%) |
| | Two or more races | 2 | (5%) |
| Diagnosis - no. (%) | Krabbe Disease | 13 | (30%) |
| | Metachromatic Leukodystrophy | 7 | (16%) |
| | Sickle Cell Disease | 3 | (7%) |
| | Gaucher Disease | 2 | (5%) |
| | Hunter Syndrome (MPS II) | 2 | (5%) |
| | MHC Class II Deficiency | 2 | (5%) |
| | Osteopetrosis | 2 | (5%) |
| | Sickle Cell Disease | 2 | (5%) |
| | XLP2 | 2 | (5%) |
| | β-Thalassemia | 1 | (2%) |
| | Cartilage-Hair Hypoplasia | 1 | (2%) |
| | Chediak-Higashi syndrome | 1 | (2%) |
| | Combined Immunodeficiency with | 1 | (2%) |
| | Multiple Intestinal Atresias | | |
| | Diamond-Blackfan Anemia | 1 | (2%) |
| | GM3 Synthase Deficiency | 1 | (2%) |
| | Hemophagocytic Lymphohistiocytosis | 1 | (2%) |
| | Hurler Syndrome (MPS IH) | 1 | (2%) |
| | Severe Congenital Neutropenia | 1 | (2%) |
| | Tay-Sachs Disease | 1 | (2%) |
| | X-linked Adrenoleukodystrophy | 1 | (2%) |
| CMV Serostatus - no. (%) | Seropositive | 16 | (36%) |
| Graft Characteristics | | | |
| HLA Match (of 6) - no. (%) | 6 | 16 | (36%) |
| | 5 | 19 | (43%) |
| | 4 | 9 | (21%) |
| HLA Match (of 8) - no. (%) | 8 | 8 | (18%) |
| | 7 | 10 | (23%) |
| | 6 | 14 | (32%) |
| | 5 | 10 | (23%) |
| | 4 | 2 | (5%) |
| TNC/kg (×10⁷) - median (range) | | 9.1 | (2.3-24.8) |
| CD34/kg (×10⁵) - median (range) | | 3.31 | (0.92-9.24) |
| Gender Mismatch - no. (%) | | 22 | (50%) |
| ABO Mismatch - no. (%) | | 28 | (64%) |

### Donors

Umbilical cord blood grafts were selected based on HLA-A and HLA-B intermediate-resolution and HLA-DRB 1 allele level typing. Unit selection was based on intermediate-resolution HLA typing with 5 of 6 matches preferred over 4 of 6 and targeting a pre-cryopreserved total nucleated cell (TNC) and CD34⁺ cell dose ≥3×10⁷/kg and ≥1.5×10⁵/kg, respectively. For IEM patients where it was feasible, candidate units meeting these parameters were evaluated for enzyme activity, and units with low enzyme activity were excluded. Donor-specific antibodies were tested in 31 patients.

### Conditioning Regimen

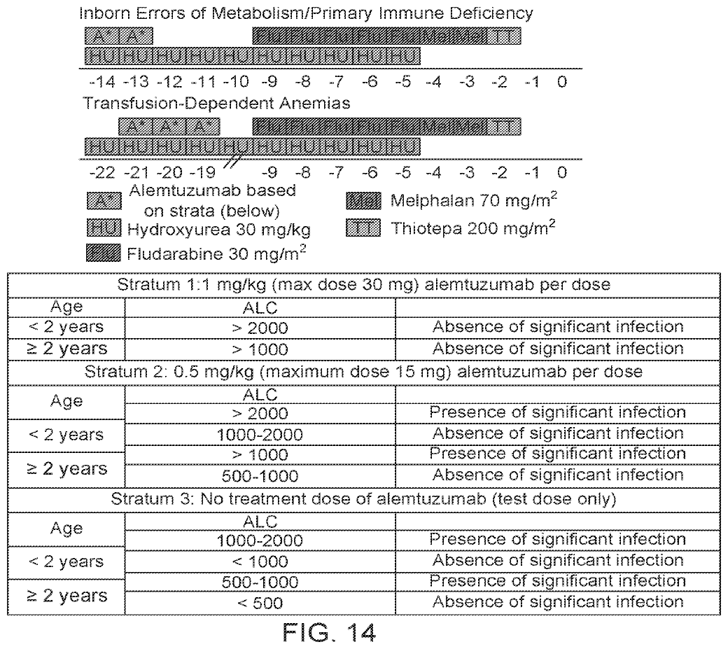

Risk-stratified alemtuzumab dosing was 0, 0.5, or 1 mg/kg intravenous (IV) on day −13 following a test dose for tolerability of 0.2 mg/kg on day −14. Patients with underlying lymphopenic conditions and/or pre-existing viral infections at risk to recur were stratified to lower alemtuzumab dosing, as shown in FIG. 14. Oral hydroxyurea (30 mg/kg/day) started between day −20 and −15 through day −5; fludarabine (30 mg/m²/day or 1 mg/kg/day, whichever dose was lower) IV started on days −9 to −5; melphalan (70 mg/m²/day) IV started on days −4 to −3; and thiotepa (200 mg/m²) started on day −2. Patients with transfusion-dependent anemias started hydroxyurea on day −22 and received alemtuzumab with a test dose on day −21 and dose per stratum on days −20 and −19 (FIG. 14).

### Graft-Versus-Host Disease (GVHD) Prophylaxis

All patients received GVHD prophylaxis with tacrolimus (continuous infusion target level: 12-16 ng/mL) and mycophenolate mofetil (MMF) 15 mg/kg IV every eight hours from day −3. Tacrolimus was converted to twice daily oral dosing by the time of discharge with target levels 8-12 ng/mL. In patients without active GVHD, MMF was discontinued after day +28, and tacrolimus taper was initiated at day +100 in patients without GVHD. Those with active viral infections were permitted to undergo faster taper of MMF. Acute GVHD was graded according to 1994 consensus criteria.

### Supportive Care

Patients were hospitalized prior to alemtuzumab infusion. Fungal prophylaxis with IV caspofungin was replaced with oral voriconazole near discharge. Bacterial prophylaxis with levofloxacin was given starting on day 0. After a certain date of the clinical trial, the protocol was revised such that all patients with cytomegalovirus (CMV) seropositivity and/or CMV PCR positivity received ganciclovir prophylaxis from day −12 to −1, followed by acyclovir or foscarnet daily. Patients at risk for herpes simplex virus (HSV) or varicella zoster virus (VZV) received acyclovir from day +1. Beginning after alemtuzumab administration, viral monitoring was performed twice weekly for adenovirus, 1-2 times weekly for CMV, and every 2 weeks for Epstein-Barr virus (EBV) with pharmacological intervention upon confirmed viremia. Pneumocystis jirovecii prophylaxis was with trimethoprim-sulfamethoxazole pre-transplant, and IV pentamidine after day +28. Patients received intravenous immunoglobulin (IVIG) 500 mg/kg IV every 2 weeks post-alemtuzumab until month 2, and for an IgG level of <500 mg/dL thereafter. Initially, the clinical trial protocol involved giving low-dose heparin infusion and ursodiol for veno-occlusive disease (VOD) prophylaxis (n=36); after that date of the clinical trial, the protocol was revised such that ursodiol alone was used for VOD prophylaxis (n=8). After a certain date of the clinical trial, the protocol was revised such that patients under 36 months of age received a single dose of rituximab near discharge for prevention of autoimmune hemolytic anemia (AIHA).

### Cord-Blood Transplantation and Donor Lymphocyte Infusion

The frozen umbilical cord blood was thawed and washed using Plasma-lyte A and centrifugation. After a certain date of the clinical trial, the protocol was revised such that umbilical cord blood washing post-thaw was not performed for patients weighing >10 kg to minimize cell loss.

When washing was performed, the pelleted cells were resuspended in 100-110 mL of cold dextran 40/albumin. When washing was not performed, the contents of the thawed umbilical cord blood were diluted to 100-110 mL of cold dextran/albumin.

In both cases, an aliquot of 5 mL from the 100-110 mL umbilical cord blood preparation was re-cryopreserved for possible future use as a donor lymphocyte infusion (cDLI).

When a recipient was administered a cDLI, the cDLI aliquot for that recipient was diluted to 30-35 mL and administered following informed consent.

Initial umbilical cord blood grafts and subsequent cDLI's were infused over 45-60 minutes.

### Post-Transplant Assessments

Chimerism was performed by the short tandem repeat method in whole blood and CD3⁺ fractions while peripheral blood lymphocyte reconstitution was monitored by flow cytometry. Immune reconstitution was assessed before conditioning and monitored post-transplant and was compared to the lower limit of age appropriate normal range for CD3, CD4, CD8, CD4/CD45RA/CD62L⁺ (surrogate recent thymic emigrants (RTE)) and NK cell numbers.

### Research Immune Studies

T cell receptor (TCR) excision circles (TREC) were measured using real-time PCR. TCR repertoire diversity was determined by quantifying TCRβ CDR3 size variability via spectratyping as described elsewhere (Chen et al., Blood, 105(2):886-93 (2005)). Normal TCRO CDR3 size was characterized as a Gaussian distribution, containing 8-10 peaks for each V3 subfamily. Plasma samples (day 0) were frozen at −80° C. until alemtuzumab measurement.

### Statistical Methods

Using the GraphPad Prism 7 software, continuous variables were analyzed using the Mann-Whitney test, while categorical variables were analyzed using Fischer's exact test or Chi-square analysis. Survival and cumulative incidence curves were calculated using non-competing risk Kaplan-Meier method and compared using the log-rank (Mantel-Cox) test. For purposes of calculating event-free survival (EFS), events were defined as death or graft failure.

### Patient Characteristics

Patient demographics are shown in Table 4. The median age was 1.7 years of age. Sixty-one percent of patients were male, and 66% were Caucasian. Most patients (68%) had newly diagnosed leukodystrophies or other IEM. All but one patient with IEM exhibited progressive neurodevelopmental deterioration prior to transplant. Sixteen (36%) patients were CMV seropositive. Five patients were receiving supplemental IgG prior to transplant, and an additional six patients were less than 12 months of age at time of transplant, when maternal CMV antibodies may be detectable. The median length of stay was 43 days (range: 27-148 days).

### Graft Characteristics

The infused, post-thaw median total nucleated cell (TNC) and CD34⁺ cell doses were 9.1×10⁷/kg (range: 2.3-24.8×10⁷/kg) and 3.31×10⁵/kg (range: 0.92-9.24×10⁵/kg), respectively. Most umbilical cord blood grafts were HLA-mismatched, and more than half were mismatched for gender and/or ABO blood type (Table 4).

### Neutrophil and Platelet Engraftment

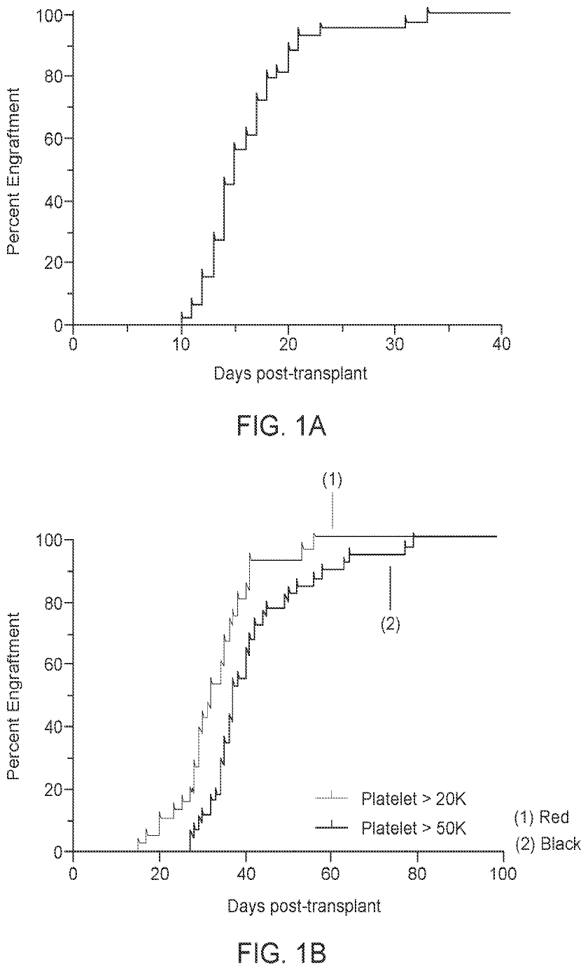

All 44 patients were engrafted with donor neutrophils at a median of 15 days post-transplant (range: 10-33) (FIG. 1A). One patient experienced secondary graft-failure on day 38 post-UCBT and underwent a successful second UCBT within three months using busulfan-based reduced toxicity conditioning. In 36 evaluable patients, median platelet engraftment ≥20,000/μL was at 32 days post-transplant (range: 15-56) (FIG. 1). Eight patients were unevaluable due to a higher platelet transfusion threshold (bleeding (N=3), sickle cell disease (N=3), platelet aggregation defect (N=1)), and one due to secondary graft failure. In 42 evaluable patients, median platelet engraftment ≥50,000/μL occurred 37 days post-transplantation (range: 27-79; FIG. 1i). Two patients were unevaluable due to requiring higher thresholds (bleeding (N=1) or graft failure (N=1)). Donor-specific anti-HLA antibodies (DSA) were tested in 31 patients, and negative in 30. The only patient with positive DSA remained fully donor at ≥3 years post-transplant. Both patients with osteopetrosis and patients with transfusion dependent anemia engrafted.

### Donor Chimerism

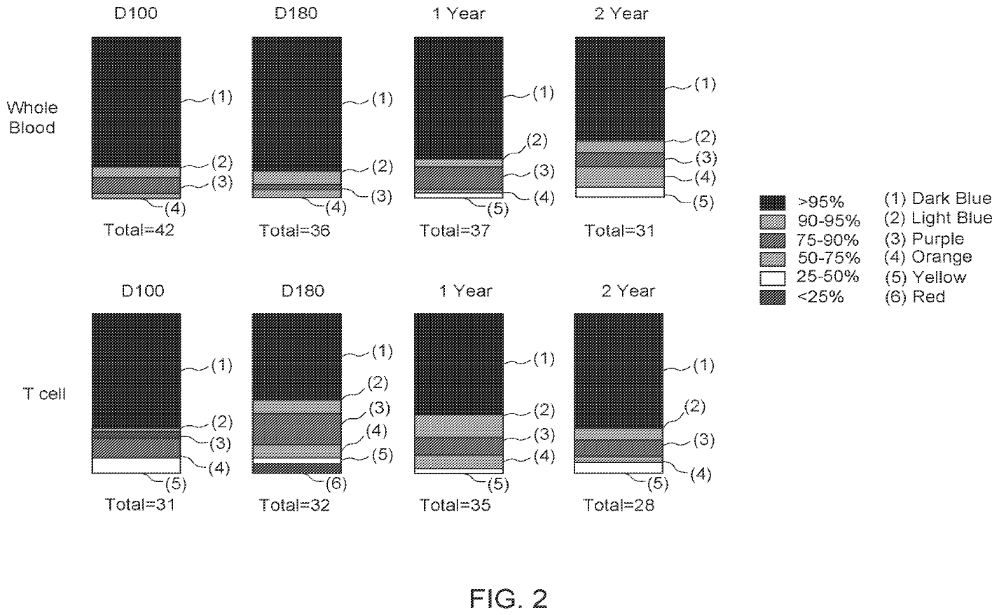

Whole blood chimerism was ≥90% donor in 37 of 42 patients at 100 days, 33 of 38 patients at 180 days, 30 of 37 patients at 1 year, and 22 of 31 patients at 2 years post-transplant. The number of patients with CD3 chimerism of ≥90% donor were similar (23 of 31 patients at 100 days, 20 of 32 patients at 180 days, 27 of 35 patients at 1 year, and 22 of 28 patients at 2 years post-transplant (Table 5, FIG. 2)). Among patients with mixed (<95% donor) whole-blood chimerism at 1 year, the median donor contribution to hematopoiesis was 85% (range 47-91%).

TABLE 5
Complications post-transplant.

| Complication | | N = 44 | |
|---|---|---|---|
| Engraftment Syndrome | | 16 | (36%) |
| Acute GVHD - no. (%) | | 21 | (48%) |
| Grade I - no. (% of GVHD) | | 9 | (43%) |
| | Skin Only | 9 | (43%) |
| Grade II - no. (% of GVHD) | | 7 | (33%) |
| | Skin Only | 6 | (29%) |
| | Skin + GI | 1 | (5%) |
| Grade III - no. (% of GVHD) | | 5 | (24%) |
| | Skin + GI | 2 | (10%) |
| | GI only | 3 | (14%) |
| Grade IV - no. (% of GVHD) | | 0 | (0%) |
| GVHD grade I-IV onset day - | | 47 | (13-145) |
| median (range) | | | |
| GVHD grade II-IV onset day - | | 39.5 | (14-145) |
| median (range) | | | |
| Whole blood chimerism | >95% | 28 | (76%) |
| (donor percentage) at 1 yr | | | |
| (n = 37) - no. (%) | | | |
| | 90-95% | 2 | (5%) |
| | 75-89% | 5 | (14%) |
| | 50-74% | 1 | (3%) |
| | 25-49% | 1 | (3%) |
| | <25% | 0 | (0%) |
| T cell chimerism (donor | >95% | 22 | (63%) |
| percentage) at 1 yr | | | |
| (n = 35) - no. (%) | | | |
| | 90-95% | 5 | (14%) |
| | 75-89% | 4 | (11%) |
| | 50-74% | 3 | (9%) |
| | 25-49% | 1 | (3%) |
| | <25% | 0 | (0%) |
| Opportunistic Infections | | 27 | (61%) |
| at 1 year - no. (%) | | | |
| | Adenovirus - no. (% of infections) | 11 | (41%) |
| | EBV - no. (% of infections) | 10 | (37%) |
| | HHV-6 - no. (% of infections) | 9 | (33%) |
| | CMV - no. (% of infections) | 6 | (22%) |
| | Mycobacteria - no. (% of infections) | 1 | (4%) |
| | Fungal - no. (% of infections) | 1 | (4%) |

### Transplant Related Morbidity

No patients experienced transplantation associated microangiopathy, interstitial pneumonitis syndrome (TPS), pericardial effusion requiring medical or surgical interventions, or veno-occlusive disease (VOD). Additionally, no patients experienced macroscopic hematuria or hemorrhagic cystitis. Autoimmune hemolytic anemia (AIHA) occurred in ten patients, seven of whom received an ABO mismatched graft. One patient required systemic corticosteroids following prompt upfront rituximab therapy.

### GVHD

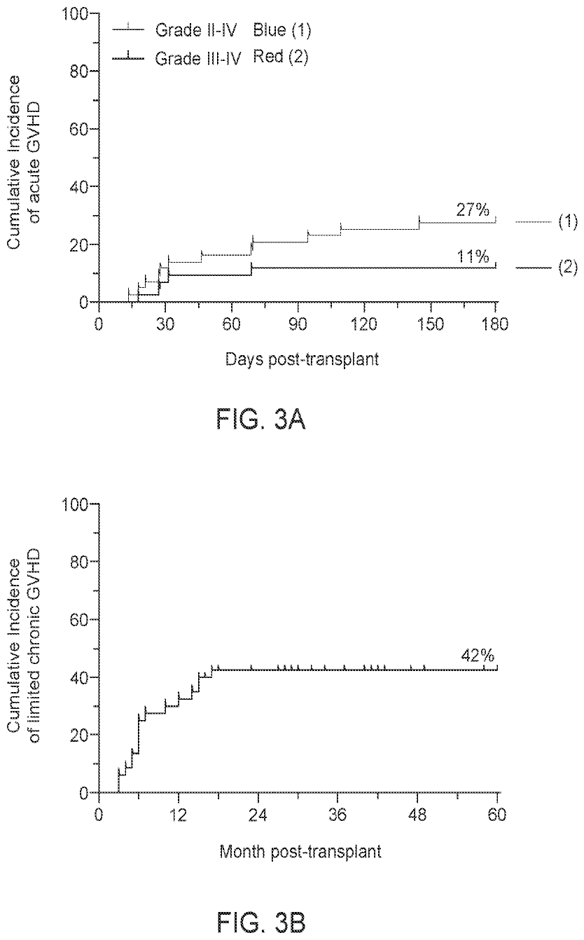

Engraftment syndrome associated with non-infectious fever, rash, and capillary leak necessitating corticosteroids was seen in 7 subjects (16%) at a median of 11 days post-transplant (range: 8-13). The cumulative incidence of grades II-IV and III-IV acute GVHD by day 180 was 27% (95% CI: 10-48%) and 11% (95% CI: 1-40%), respectively (FIG. 3A). The median day of grade II-IV acute GVHD onset was 40 days post-transplant (range: 14-145). Skin was the most frequently involved organ (Table 5). The cumulative incidence of any chronic GVHD was 42% (95% CI: 25-58%; FIG. 3B), and all were limited to mild skin rash controlled with topical steroid therapy. No patient developed severe/extensive chronic GVHD. Importantly, 72% of those who developed chronic GVHD did so during taper of immunosuppression and were easily controlled with topical therapy and slowing the taper or stepping back one dose level.

### Survival

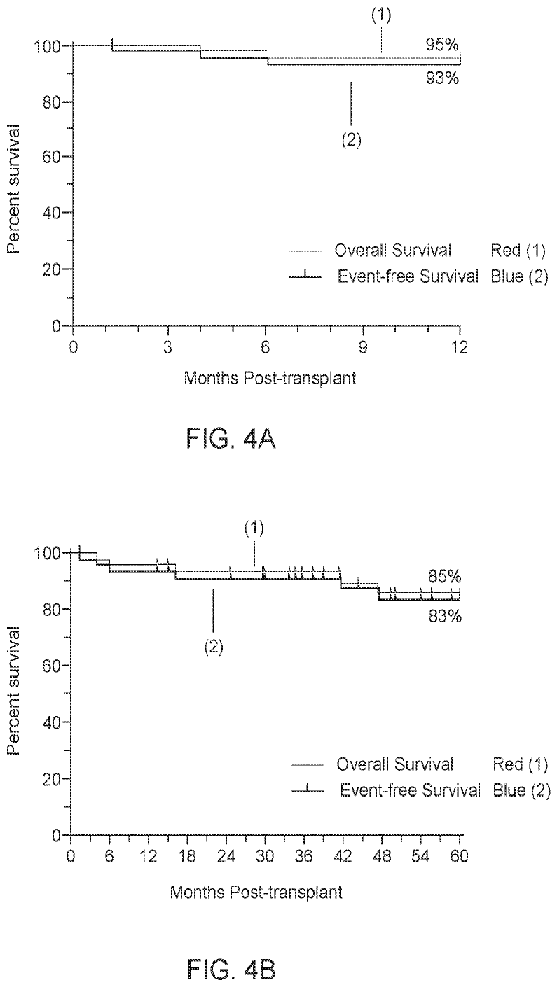

The median follow-up of surviving patients is 49.5 months (range: 13.3-98.4). Overall survival at 1 and 5 years post-transplant is 95% (95% CI: 83-99%) and 85% (95% CI: 67-94%), respectively. EFS at 1 and 5 years post-transplant is 93% (95% CI: 80-98%) and 83% (95% CI: 66-92%), respectively (FIG. 4A, B). Treatment-related mortality was 5% (95% CI: 0-46%) at 1 year post-transplant. Two patients died prior to 1 year; one from adenoviral/CMV/Parainfluenza disease at day +121, and one from progression of Tay-Sachs disease complicated by adenoviral pancreatitis at day +185 post-transplant. Three additional patients (neurotopic Gaucher disease, N=2; metachromatic leukodystrophy (MILD), N=1) died at 15, 41, and 47 months post-transplant. All three of these patients were significantly affected by their IEM prior to transplantation and died from complications of their pre-transplant comorbidities.

### Enzyme Activity and Neurodevelopmental Outcomes

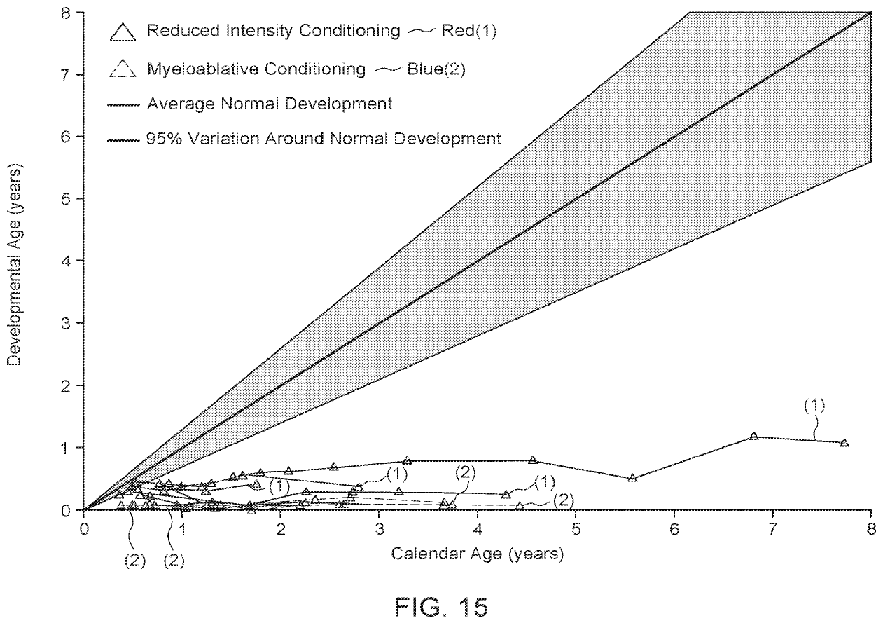

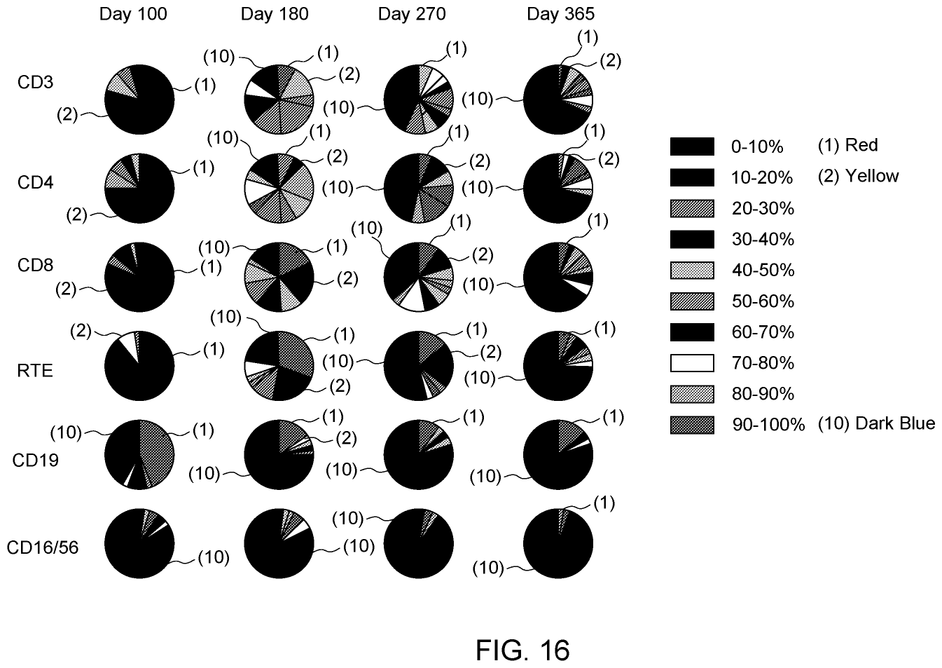

In the subset of patients with IEM with enzyme activities measured, 16 of 16 at 100 days post-transplant, 15 of 15 at 180 days post-transplant, and 14 of 14 at 1 year post-transplant had enzyme activities in the unaffected range. There were four symptomatic infantile Krabbe patients, in stage 2 of disease progression. These patients stabilized at a low functional level and did better than historical disease-stage matched controls receiving myeloablative conditioning (FIG. 15). The two symptomatic late infantile Krabbe patients stabilized in the motor area and continue to gain cognitive skills. Two of the symptomatic juvenile Krabbe patients are walking and attending regular school, and the third progressed and is functioning at a low level. Of the two late-infantile onset symptomatic MILD patients, one is able to walk with a walker, and the other died in the peri-transplant period of infection. Four of the juvenile-onset MILD patients are able to walk, speak, and function in the delayed range but are stable. The asymptomatic adrenoleukodystrophy patient is able to walk and talk in spite of his behavioral difficulties and developmental delay related to the additional diagnosis of autism. One patient with Hurler syndrome transplanted at 22 months of age and two patients with Hunter Syndrome transplanted at 10 months and 1.25 years of age continue to gain skills but have mild global developmental delay.

### Immune Reconstitution

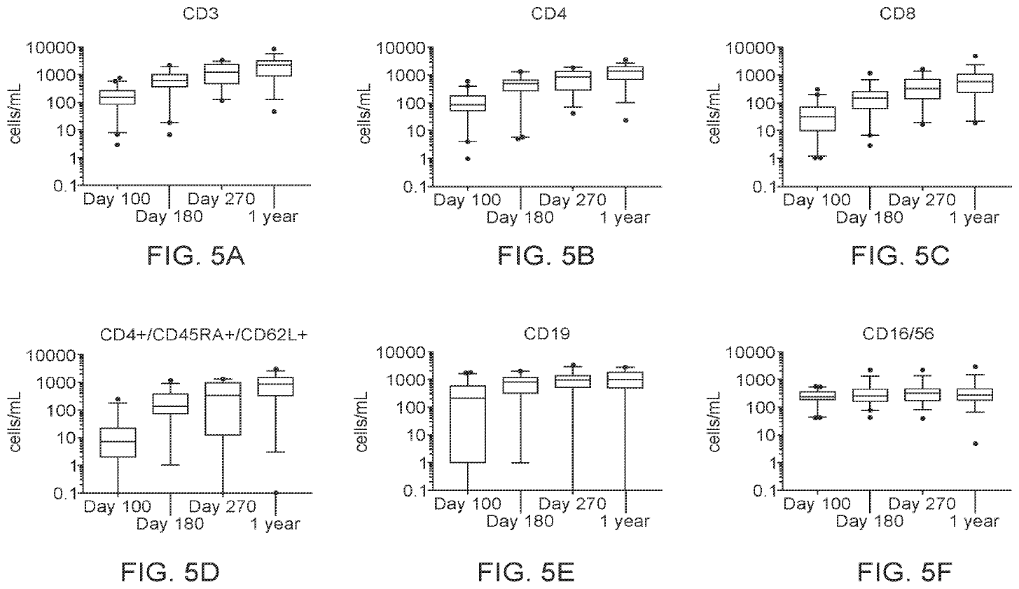

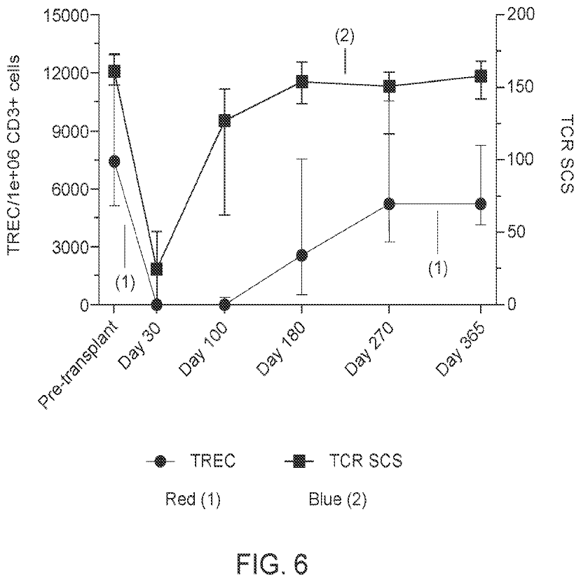

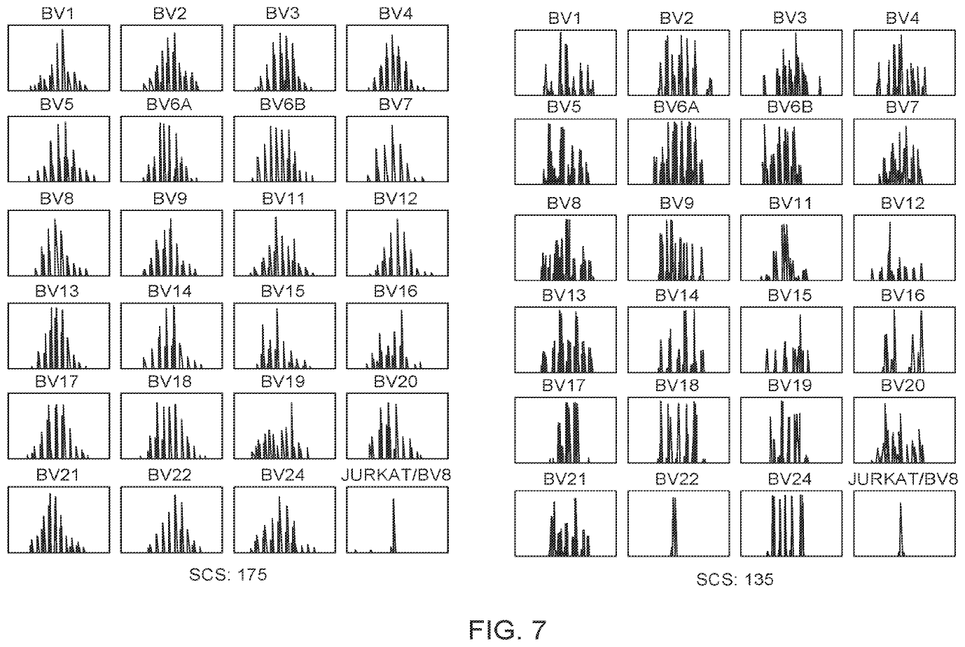

NK and B lymphocytes achieved normal numbers by day 100 post-transplant, while T cell reconstitution was slower, accelerating between day 100 and 180 post-transplant (FIGS. 5A-F, 16). TREC and TCR V3 repertoire were measured to better assess thymopoiesis. TCR V3 repertoire was near pre-transplant values by 180 days post-transplant, while TREC copies began to rise by 100 days post-transplant reaching normal values around 270 days post-transplant concordant with flow cytometry values for RTE (FIGS. 6, 7). Twenty-eight of the 38 engrafted and evaluable surviving patients (74%) were off systemic immunosuppression at 1 year post-transplant, and 30 of 38 patients (79%) did not require IgG supplementation at 1 year post-transplant.

### Infections

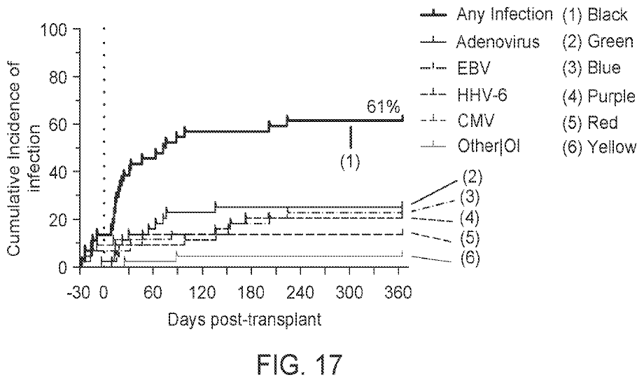

Following conditioning, the cumulative incidence of first opportunistic infection (01) at 1 year post-transplant was 61% (95% CI: 49-72%) with a median onset day of 18 days post-transplant (range: −27-224; FIG. 17, Table 5). Eight patients had infections with more than one organism. The majority of OI were asymptomatic viremia. One patient developing Candida parapsilosis infection at day +24, and another was found to have asymptomatic pulmonary Mycobacterium kansasii infection on screening chest X-ray at 3 months visit. The majority of patients received appropriate pharmacologic therapy for viremia. The patient with congenital intestinal atresia developed EBV-positive plasmacytoma 6 months after small bowel transplant and approximately 2 years post-UCBT and is alive and in remission over 5 years post-UCBT. Two patients developed refractory adenoviremia and were treated with virus-specific T cells under an investigational new drug protocol and died.

### Alemtuzumab Levels

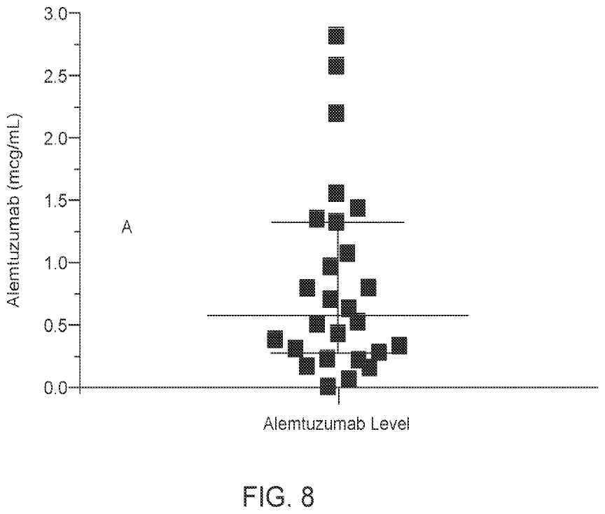

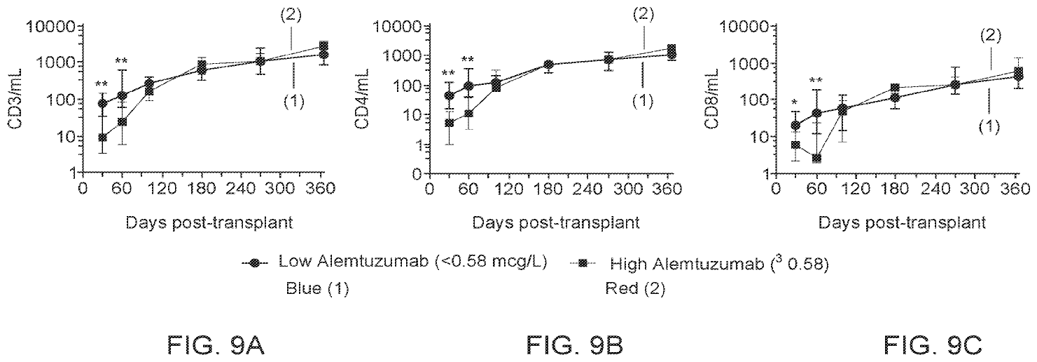

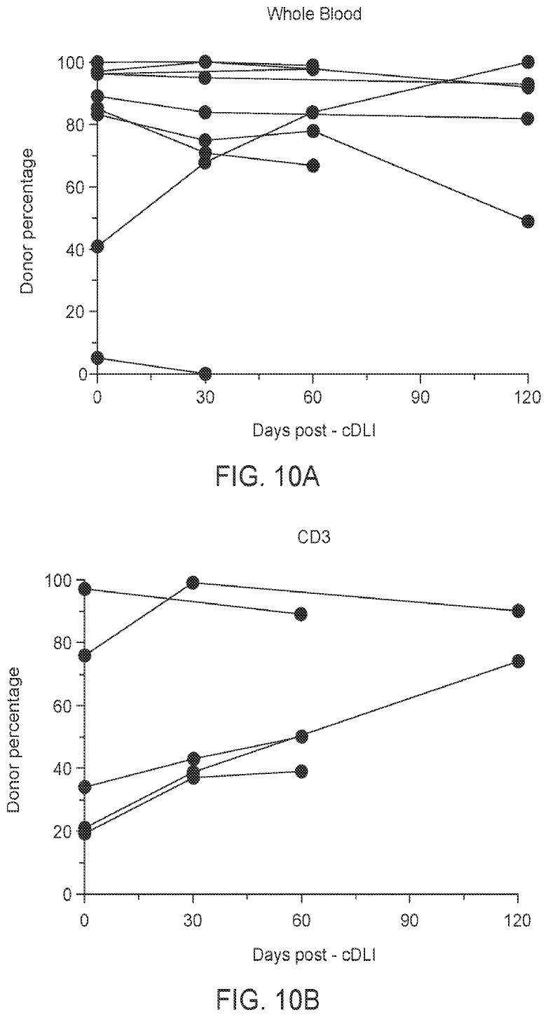

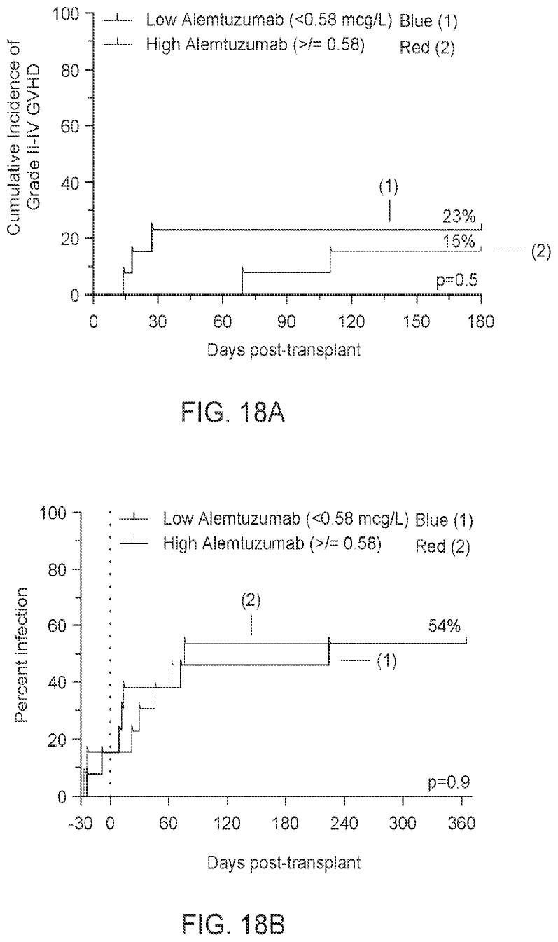

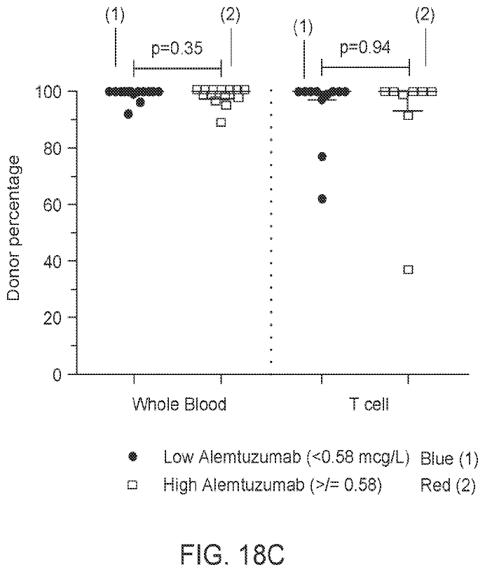

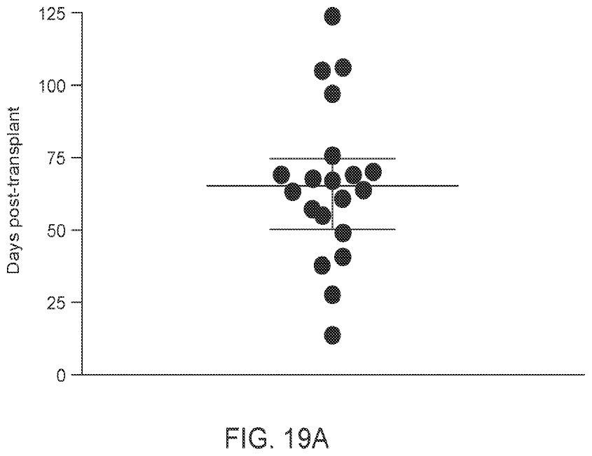

Most patients (N=39) received a cumulative dose of 1.2 mg/kg of alemtuzumab. One patient received 0.7 mg/kg due to active CMV viremia and parainfluenza at the time of conditioning, while four patients (one with HLH and three with hemoglobinopathies) received 2.2 mg/kg. Twenty-six patients had samples drawn on the day of transplant for alemtuzumab levels. The median alemtuzumab level was 0.58 μg/mL (range: 0-2.82 μg/mL; FIG. 8). Notably, patients exhibiting day 0 levels above the median had fewer circulating T cells at days +30 and +60 post-transplant, demonstrating greater early lymphotoxicity with resolution by day 100 (FIG. 7A-C). At 1 year post-transplant, there was no significant difference in GVHD, infection, or mixed chimerism in the whole blood or T cell fractions between patients with high or low day 0 alemtuzumab levels (FIG. 18A-C).

### Cord-Derived Donor Lymphocyte Infusion

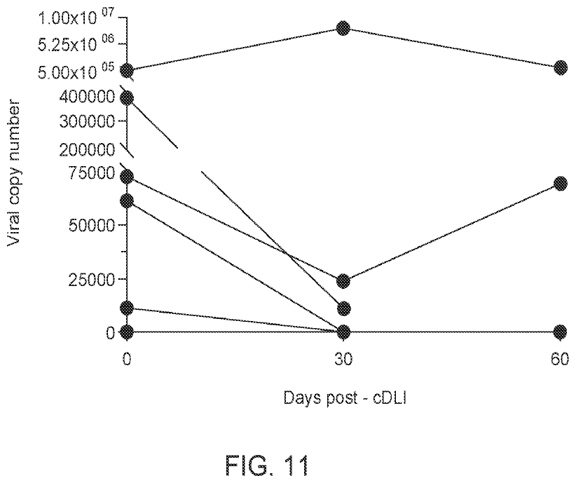

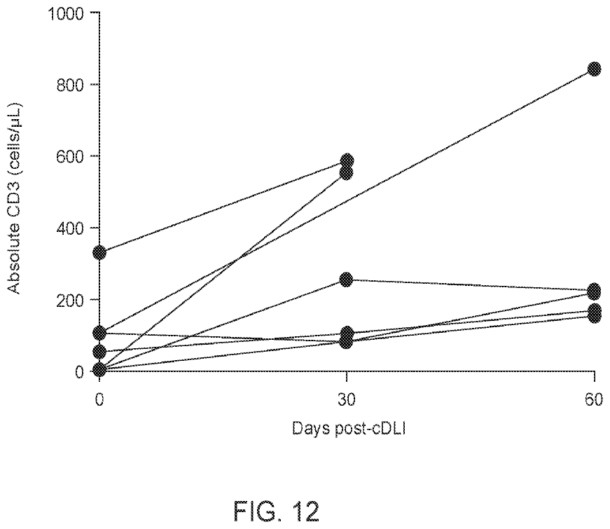

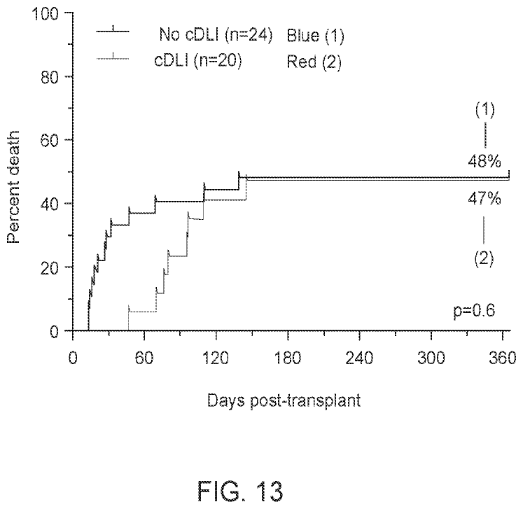

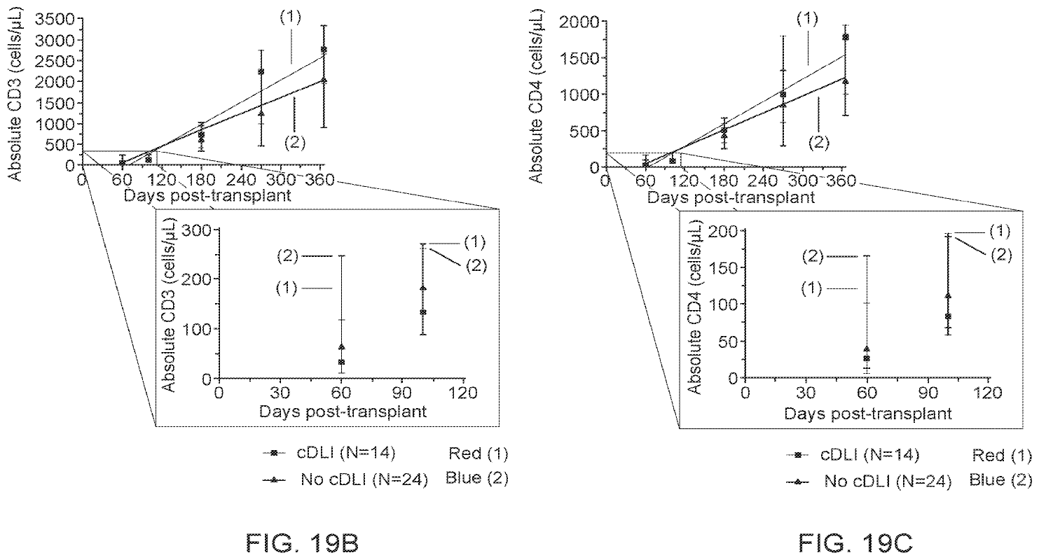

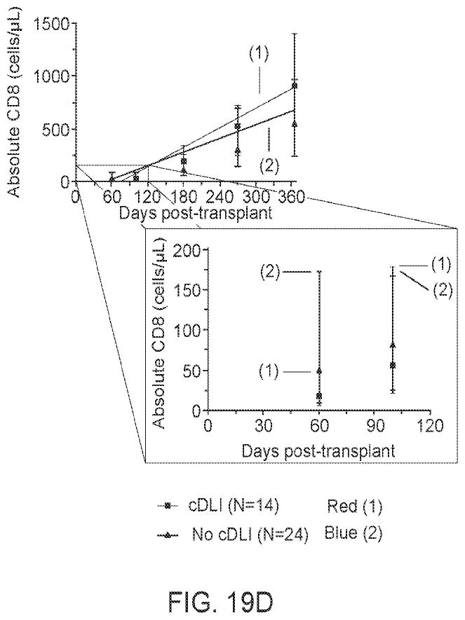

Bone marrow transplant physicians were offered three clinical criteria to use cDLI: those with potentially life-threatening viremia or viral infections (N=10), mixed T cell chimerism (N=9), or sluggish T cell reconstitution (<100/μL after 2 months, N=7). Seven of the 20 cDLI recipients had more than one indication for cDLI, most commonly infection with delayed CD3 recovery (N=4). Twenty patients received cDLI at a median of 66 days post-transplant (range: 14-124) to foster homeostatic T cell expansion in the first 4-6 months until effective thymopoiesis, with the majority (n=14) receiving cDLI between day 38-75 (FIG. 19A). The two patients who received cDLI prior to day 30 were given due to very high-grade adenoviremia and represented the only patients who experienced transplant-related mortality, dying despite additional virus specific third party cDLI. Alemtuzumab dose de-escalation based on prior viral infection was introduced after these early back to back subjects. The median cell dose of the cDLI was 7.56×10⁵ CD3/kg (range: 2.36-24.5×10⁵) with negligible CD34⁺ content (median: 1.58×10⁴/kg; range: 0.79-5.04×10⁴/kg). Median viability post-thaw was 94% (range: 76-100%). Improvement in donor chimerism was seen in 5 of 9 patients, reduction in viral load and/or clinical symptoms of viral infection in 6 of 10 patients, and absolute CD3 counts rose in 7 of 7 patients (FIGS. 11, 12, and 13). The kinetics of CD3, CD4, CD8 cell reconstitution for subjects who received cDLI between day +38 and 75 (N=14) was examined between 2 and 12 months after UCBT and compared with those who never received cDLI (N=24; FIG. 19B-D). While the patients who received cDLI showed an increase in the rate of T cell reconstitution, it did not reach statistical significance reflecting the small cohorts and three different indications. Eight patients who received cDLI developed grade I-II skin GVHD at a median of 19 days post-cDLI (range: 4-81). Two patients with a past history of acute GVHD received cDLI without GVHD flare. No patients who received cDLI developed grade III or IV acute GVHD or severe/extensive chronic GVHD. There was no difference in the cumulative incidence of acute GVHD between those who received cDLI and those who did not (47% (95% CI: 23-69%) and 48% (95% CI: 28-66%), respectively; p=0.6); FIG. 13).

These results demonstrate that an initial portion (e.g., about 95 percent) of a frozen umbilical cord blood preparation (e.g., obtained from an umbilical cord blood bank) can be administered to a mammal (e.g., a human) at a first time point to promote reconstitution of an immune system of the donor in the recipient and that a subsequent portion (e.g., the remaining 5 percent) of that same umbilical cord blood preparation, which was refrozen, can be administered to that same recipient to boost the recipient's immune system at a second time point when the concentration of lymphotoxic agent(s) (e.g., alemtuzumab and/or ATG) used to originally deplete the recipient's native immune system within the recipient (or used in a de-escalation protocol) is lower.

### Example 2—Treating a Human Having a Leukodystrophy Syndrome Such as Krabbe Disease, Metachromatic Leukodystrophjy (MLD) or Adrenoleukodystrophy (ALD), or MPS Syndromes Such as Hurler Syndrome, Hunter Syndrome, or Another Inborn Errors of Metabolism

A human patient of 0.5 years and 4 years of age is identified as having a leukodystrophy syndrome and is assessed to identify an appropriate HLA match for an umbilical cord blood transplant. The human patient is prepared using an RIC regime. Once the human patient is prepared, the appropriate umbilical cord blood preparation is thawed and diluted to a total volume of 100 mL. 95 mL of that diluted umbilical cord blood preparation is administered intravenously over 45 to 60 minutes.

The remaining 5 mL of the umbilical cord blood preparation are refrozen for subsequent administration to the human patient.

After 30 to 90 days from the initial administration of the first portion, the refrozen umbilical cord blood preparation that remained is thawed and diluted to a total volume of 30-35 mL, which is administered intravenously over 45 to 60 minutes to the human patient.

The combination of administering a first portion of an umbilical cord blood preparation and a second portion of the same umbilical cord blood preparation to the human patient with the appropriate time between the first and second administration is designed to allow the human patient to establish the donor's immune system (or a chimeric donor/recipient immune system) quicker, more effectively, and/or with reduced adverse events.

### Example 3—Treating a Human Having a Bone Marrow Failure Syndrome Such as Multilineage as Aplastic Anemia or Reticular Dysgenesis, or a Red Cell Lineage/Single Lineage Defect Such as Thalassemia, Sickle Cell Disease, or Diamond Blackfan Anemia

A human patient of 0.5 years and 4 years of age is identified as having a bone marrow failure syndrome and is assessed to identify an appropriate HLA match for an umbilical cord blood transplant. The human patient is prepared using an RIC regime. Once the human patient is prepared, the appropriate umbilical cord blood preparation is thawed and diluted to a total volume of 100 mL. 95 mL of that diluted umbilical cord blood preparation is administered intravenously over 45 to 60 minutes.

The remaining 5 mL of the umbilical cord blood preparation are refrozen for subsequent administration to the human patient.

After 30 to 90 days from the initial administration of the first portion, the refrozen umbilical cord blood preparation that remained is thawed and diluted to a total volume of 30-35 mL, which is administered intravenously over 45 to 60 minutes to the human patient.

The combination of administering a first portion of an umbilical cord blood preparation and a second portion of the same umbilical cord blood preparation to the human patient with the appropriate time between the first and second administration is designed to allow the human patient to establish the donor's immune system (or a chimeric donor/recipient immune system) quicker, more effectively, and/or with reduced adverse events.

### Example 4—Treating a Human Having a PID Syndrome Such as Wiskott-Aldrich Syndrome, an HLH Syndrome, or a Syndrome with CGD, LAD, and/or JAK/STAT Signaling Defects

A human patient of 0.5 years and 4 years of age is identified as having a PID syndrome and is assessed to identify an appropriate HLA match for an umbilical cord blood transplant. The human patient is prepared using an RIC regime. Once the human patient is prepared, the appropriate umbilical cord blood preparation is thawed and diluted to a total volume of 100 mL. 95 mL of that diluted umbilical cord blood preparation is administered intravenously over 45 to 60 minutes.

The remaining 5 mL of the umbilical cord blood preparation are refrozen for subsequent administration to the human patient.

After 30 to 90 days from the initial administration of the first portion, the refrozen umbilical cord blood preparation that remained is thawed and diluted to a total volume of 30-35 mL, which is administered intravenously over 45 to 60 minutes to the human patient.

The combination of administering a first portion of an umbilical cord blood preparation and a second portion of the same umbilical cord blood preparation to the human patient with the appropriate time between the first and second administration is designed to allow the human patient to establish the donor's immune system (or a chimeric donor/recipient immune system) quicker, more effectively, and/or with reduced adverse events.

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

1. A method for providing a mammal with a hematopoietic stem cell transplantation, wherein said method comprises:
(a) administering a first portion of an umbilical cord blood preparation obtained from a single donor to said mammal at a first time point, and
(b) administering a second portion of said umbilical cord blood preparation to said mammal at a second time point, wherein said second time point is two weeks to eight months after said first time point.

2. The method of claim 1, wherein said mammal is a human.

3. The method of claim 1, wherein said first portion comprises from about 80 to about 95 percent of said umbilical cord blood preparation.

4. (canceled)

5. The method of claim 1, wherein said first portion was obtained from said umbilical cord blood preparation after said umbilical cord blood preparation was thawed from a frozen state only once.

6. The method of claim 1, wherein said method comprises refreezing a remaining portion of said umbilical cord blood preparation that remains after said first portion is obtained for said administering step (a).

7. The method of claim 1, wherein said second portion comprises from about 5 to about 20 percent of said umbilical cord blood preparation.

8. (canceled)

9. The method of claim 1, wherein said second portion was obtained from said umbilical cord blood preparation after said umbilical cord blood preparation was thawed from a frozen state twice.

10-14. (canceled)

15. A method for treating a mammal having a non-malignant disorder with a hematopoietic stem cell transplantation, wherein said method comprises:
(a) administering a first portion of an umbilical cord blood preparation obtained from a single donor to said mammal at a first time point, and
(b) administering a second portion of said umbilical cord blood preparation to said mammal at a second time point, wherein said second time point is two weeks to eight months after said first time point.

16. The method of claim 15, wherein said mammal is a human.

17. The method of claim 15, wherein said non-malignant disorder is a primary immunodeficiency disease, a hemoglobinopathy, a bone marrow failure syndrome, or an inborn errors of metabolism disorder.

18. The method of claim 15, wherein said first portion comprises from about 80 to about 95 percent of said umbilical cord blood preparation.

19. (canceled)

20. The method of claim 15, wherein said first portion was obtained from said umbilical cord blood preparation after said umbilical cord blood preparation was thawed from a frozen state only once.

21. The method of claim 15, wherein said method comprises refreezing a remaining portion of said umbilical cord blood preparation that remains after said first portion is obtained for said administering step (a).

22. The method of claim 15, wherein said second portion comprises from about 5 to about 20 percent of said umbilical cord blood preparation.

23. (canceled)

24. The method of claim 15, wherein said second portion was obtained from said umbilical cord blood preparation after said umbilical cord blood preparation was thawed from a frozen state twice.

25-29. (canceled)

30. A method for treating a mammal having a non-malignant disorder with a hematopoietic stem cell transplantation, wherein said method comprises:
(a) thawing a frozen umbilical cord blood preparation obtained from a single donor, thereby obtaining a thawed umbilical cord blood preparation,
(b) administering a first portion of said thawed umbilical cord blood preparation to said mammal at a first time point,
(c) re-freezing a remaining portion of said thawed umbilical cord blood preparation that remains after said first portion was used for said administering step (b), thereby obtaining a refrozen umbilical cord blood preparation,
(d) thawing said refrozen umbilical cord blood preparation, thereby obtaining a twice thawed umbilical cord blood preparation, and
(e) administering a portion of said twice thawed umbilical cord blood preparation to said mammal at a second time point, wherein said second time point is two weeks to eight months after said first time point.

31. The method of claim 30, wherein said mammal is a human.

32. The method of claim 30, wherein said non-malignant disorder is a primary immunodeficiency disease, a hemoglobinopathy, a bone marrow failure syndrome, or an inborn errors of metabolism disorder.

33. The method of claim 30, wherein said first portion comprises from about 80 to about 95 percent of said frozen umbilical cord blood preparation.

34. (canceled)

35. The method of claim 30, wherein said second portion comprises from about 5 to about 20 percent of said frozen umbilical cord blood preparation.

36-41. (canceled)
